(19) United States
(12) Patent Application Publication
(10) Pub. No.: US 2019/0352324 A1
(43) Pub. Date: Nov. 21, 2019

(54) NOVEL STEVIOL GLYCOSIDE, METHOD FOR PRODUCING SAME, AND SWEETENER COMPOSITION CONTAINING SAME

(71) Applicant: SUNTORY HOLDINGS LIMITED, Osaka (JP)

(72) Inventors: Kazunari IWAKI, Kyoto (JP); Katsuro MIYAGAWA, Kyoto (JP); Eiichiro ONO, Kyoto (JP); Tadayoshi HIRAI, Kyoto (JP); Misa OCHIAI, Kyoto (JP); Koji NAGAO, Kanagawa (JP); Soichiro URAI, Kanagawa (JP); Takehiro WATANABE, Kyoto (JP); Kohki FUJIKAWA, Kyoto (JP)

(73) Assignee: SUNTORY HOLDINGS LIMITED, Osaka (JP)

(21) Appl. No.: 16/473,081

(22) PCT Filed: Dec. 26, 2017
(86) PCT No.: PCT/JP2017/046806

§ 371 Date: Jun. 24, 2019

(30) Foreign Application Priority Data
Dec. 27, 2016 (JP) 2016-253248

(51) Int. Cl. C07H 15/256; A23L 2/02; A23L 27/20; A23L 2/38; A23L 27/30

(52) U.S. Cl. CPC C07H 15/256; A23L 2/02; A23L 27/33; A23L 2/38; A23L 27/2052

(57) ABSTRACT

The purpose of the present invention is to determine the structure of a novel steviol glycoside that is detected in cultivers containing an abundance of Reb.C (also called dulcoside B) and that affects the quality of taste in a small amount, and to identify the quality of taste properties. According to the present invention, a compound represented by formula (1), or a derivative, salt, or hydrate thereof is provided.

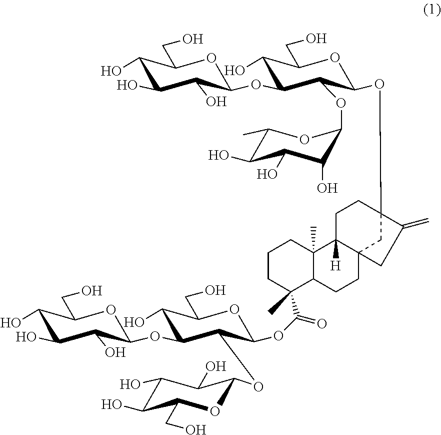

### TECHNICAL FIELD

The present invention relates to a novel steviol glycoside, a method for producing the same, and a sweetener composition containing the same. Furthermore, the present invention also relates to a food or beverage, a plant, an extract thereof and a flavor controlling agent containing the novel steviol glycoside.

### BACKGROUND ART

Leaves of Stevia rebaudiana contain a secondary metabolite called Steviol which is one type of diterpenoids, where steviol glycoside provides sweetness that is nearly 300 times the sweetness of sugar and is therefore utilized as a calorieless sweetener in the food industry. The demand for calorieless sweeteners is growing day by day as obesity has become a serious social problem worldwide and also for the sake of health promotion and reduction in the medical expenditure. Currently, aspartame and acesulfame potassium, which are artificially synthesized amino acid derivatives, are utilized as artificial sweeteners, but natural calorieless sweeteners like the steviol glycosides are expected to be safer and more likely to gain public acceptance.

The major steviol glycosides from stevia are ultimately glycosylated to a glycoside named rebaudioside A (Reb.A) that has four sugar moieties (FIG. 1). Stevioside, namely, a tri-glycosylated steviol glycoside and a precursor of Reb.A, is the most abundant glycoside. These two glycosides are the main substances responsible for the sweetness of stevia. Stevioside accounts for the largest content in stevia leaves and is known to provide sweetness that is about 250-300 times the sweetness of sugar. Reb.A is a tetra-glycosylated steviol glycoside that has strong sweetness (350-450 times sugar) with good taste quality. They have been drawing attention as calorieless sweeteners. Besides them, existence of glycosides that are considered to be reaction intermediates and analogs having different types of sugar moieties are known. For example, while all of the four glycoside sugar moieties of Reb.A are glucose, rebaudioside C (Reb.C) is known to have rhamnose instead of glucose attached to C-2 of glucose at C-13, and rebaudioside F (Reb.F) is known to have xylose attached at the same position.

To date, attempts have been made to obtain a stevia plant having a higher Reb.A content than wild-type stevia plants by breeding or the like since taste quality of Reb.A, in which all of the four glycoside sugar moieties are glucose, is good (for example, Patent document 1).

### PRIOR ART DOCUMENT

### Patent Document

Patent document 1: Japanese Patent No. 3436317

### SUMMARY OF INVENTION

### Problem to be Solved by the Invention

Meanwhile, some of the stevia cultivers resulting from breeding may contain a minute amount of a steviol glycoside whose structure is not yet identified, where the presence of such steviol glycoside present in minute quantity may potentially be contributing to the flavor characteristic of the stevia extract. Moreover, although researches have been made thus far on steviol glycosides obtained by further attaching glucose to Reb.A and on cultivers containing the same, not much research has been made at this point on a cultiver containing an abundant amount of a steviol glycoside having rhamnose like Reb.C and on such a steviol glycoside.

Accordingly, the objective of the present invention is to determine the structure of a novel steviol glycoside present in minute quantity that affects the taste quality, and to identify the characteristics of its taste quality. In addition, further objectives of the present invention are to provide a novel steviol glycoside, a method for producing the same, and a sweetener composition containing the same.

### Means for Solving the Problem

The present inventors have gone through extensive investigation to solve the above-described problem, and as a result of which succeeded in determining the structure of the novel steviol glycoside present in minute quantity that affects the taste quality. The present invention was made based on the above-described finding.

### Effect of the Invention

The present invention can provide a novel steviol glycoside present in minute quantity that affects the taste quality. Furthermore, the present invention can also provide a method for producing the novel steviol glycoside, and a sweetener composition, a food or beverage, a plant, an extract thereof and a flavor controlling agent containing the novel steviol glycoside.

### BRIEF DESCRIPTION OF DRAWINGS

FIG. 1 illustrates a diagram showing structures and names of steviol glycosides.

FIG. 2 illustrates a diagram showing a selected ion chromatogram of Sample 1 at m/z of 1273.5.

FIG. 3 illustrates diagrams showing MS/MS and MS³ fragmented mass spectra of rebaudioside N and the compound represented by Formula (1).

FIG. 4 illustrates (a) a diagram showing a ¹H-NMR spectrum of Compound 11 (800 MHz, Pyr-d5); and (b) a diagram showing a ¹³C-NMR spectrum of Compound 11 (200 MHz, Pyr-d5).

FIG. 5 illustrates (a) a diagram showing a ¹H-¹H cosy spectrum of Compound 11 (800 MHz, Pyr-d5): and (b) a diagram showing a HSQC spectrum of Compound 11 (800 MHz, Pyr-d5).

FIG. 6 illustrates diagrams showing a procedure for identifying a novel steviol glycoside contained in an extract of a plant.

FIG. 7 illustrates a diagram showing a selected ion chromatogram of a sample obtained by biosynthesis at m/z of 1273.5.

FIG. 8 illustrates diagrams showing results of sensory evaluations for comparison between the novel steviol glycoside and rebaudioside A.

FIG. 9 illustrates graphs showing results from evaluating an effect of the flavor controlling agent of the present invention to improve the lingering aftertaste of Reb.A.

FIG. 10 illustrates graphs showing results from evaluating an effect of a flavor controlling agent of the present invention to improve the lingering aftertaste of Reb.D.

FIG. 11 illustrates graphs showing results from evaluating an effect of the flavor controlling agent of the present invention to enhance sweetness of sugar (sucrose).

### MODES FOR CARRYING OUT THE INVENTION

Hereinafter, the present invention will be described in detail. The following embodiment is provided for illustrating the present invention and not with the intention of limiting the present invention solely to this embodiment. The present invention may be carried out in various modes without departing from the scope thereof. All of the documents, publications, patent publications and other patent documents cited herein are incorporated herein by reference.

The terms “rebaudioside” and “Reb.” as used herein have the same meaning and both refer to “rebaudioside”. Similarly, the terms “dulcoside” and “dulcoside” as used herein have the same meaning and both refer to “dulcoside”.

### 1. Novel Steviol Glycoside

For the first time, the present inventors identified the structure of a minute amount of a novel steviol glycoside that affects taste quality. The novel steviol glycoside of the present invention (hereinafter, also referred to as the “glycoside of the present invention”) is a compound represented by Formula (1):

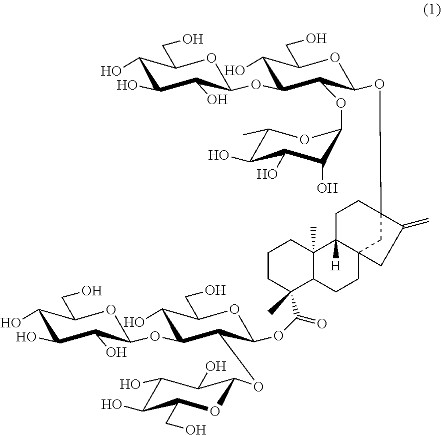

or a derivative, a salt or a hydrate thereof.

As represented above, the glycoside of the present invention has a sugar chain containing three glucose moieties at C-19 of steviol and a sugar chain containing two glucose moieties and one rhamnose moiety at C-13.

The glycoside of the present invention may not only be the compound represented by Formula (1) but may also be a derivative, a salt or a hydrate thereof. The term “derivative” as used herein refers to a compound resulting from a structural change of a minor moiety of the compound, for example, a compound in which some of the hydroxyl groups are substituted with other substituents. Therefore, derivatives of the compound represented by Formula (1) include compounds in which some of the hydroxyl groups contained in the compound have been substituted with a substituent selected from hydrogen, a halogen, an alkyl group, an alkenyl group, an alkynyl group, an aryl group, an amino group, a cyano group or the like. As used herein, a “salt of the compound represented by Formula (1)” refers to a physiologically acceptable salt, for example, a sodium salt, of the compound represented by Formula (1). Furthermore, a “hydrate of the compound represented by Formula (1)” as used herein refers to a compound resulting from addition of a water molecule to the compound represented by Formula (1).

While the glycoside of the present invention is not particularly limited, it may be a plant-derived product, a chemically synthesized product or a biosynthetic product. For example, it may be isolated and purified from a plant with abundant Reb.C, or it may be obtained by chemical synthesis or biosynthesis. Details of a method for producing a glycoside of the present invention will be described later herein.

The glycoside of the present invention is sweeter than sugar (sucrose), has taste quality of good lingering sweet aftertaste, and can affect the taste quality of foods/beverages in a small amount. Thus, the glycoside of the present invention can be used as a novel sweetener.

Moreover, in another aspect of the present invention, the novel steviol glycoside of the present invention is a compound represented by Formula (A):

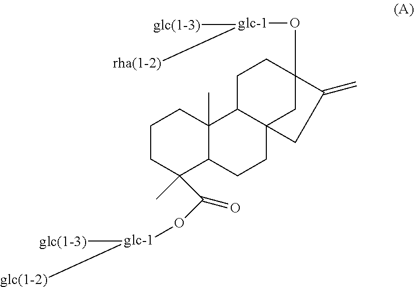

or a derivative, a salt or a hydrate thereof.

### 2. Sweetener Composition Containing Novel Steviol Glycoside

In one aspect of the present invention, a sweetener composition containing the compound represented by Formula (1), or a derivative, a salt or a hydrate thereof (hereinafter, also referred to as the “sweetener composition of the present invention”) is provided. The sweetener composition of the present invention is not particularly limited as long as it contains the compound represented by Formula (1), or a derivative, a salt or a hydrate thereof, and it may be a composition containing an extract containing the compound represented by Formula (1), or a derivative, a salt or a hydrate thereof.

The amount of the glycoside of the present invention contained in the sweetener composition of the present invention is not particularly limited.

Alternatively, the sweetener composition of the present invention is preferably a composition containing the glycoside of the present invention in a larger amount than the amount in a wild-type stevia or stevia extract by at least 0.01%. As mentioned above, the glycoside of the present invention was detected for the first time in a cultiver containing abundant Reb.C, and it is not contained in a wild-type stevia or an extract thereof at all or, if any, contained in an amount of the detection limit or less.

The sweetener composition of the present invention may further contain other steviol glycosides. For example, the sweetener composition of the present invention may contain, in addition to the glycoside of the present invention, one or more types of steviol glycosides selected from the group consisting of rebaudioside A, rebaudioside B, rebaudioside C, rebaudioside D, rebaudioside E, rebaudioside F, rebaudioside I, rebaudioside J, rebaudioside K, rebaudioside N, rebaudioside M, rebaudioside O, rebaudioside Q, rebaudioside R, dulcoside A, rubusoside, steviol, steviol monoside, steviol bioside and stevioside.

In a case where other steviol glycoside is contained, the composition ratio of the glycoside of the present invention and other steviol glycoside is preferably 0.01:9.99-6:4 in a mass ratio.

The sweetener composition of the present invention may further contain a general sweetener. Examples of such a general sweetener include natural sweeteners such as fructose, sugar, fructose-glucose syrup, glucose, maltose, sucrose, high-fructose syrup, sugar alcohol, oligosaccharide, honey, pressed sugarcane juice (brown sugar syrup), starch syrup, Lo Han Kuo (Siraitia grosvenorii) powder, Lo Han Kuo extract, licorice powder, licorice extract, Thaumatococcus daniellii seed powder and Thaumatococcus daniellii seed extract, and artificial sweeteners such as acesulfame potassium, sucralose, neotame, aspartame and saccharin. Among them, natural sweeteners are preferably used from the aspect of imparting clean taste, easy drinkability, natural flavor and moderately rich taste, where fructose, glucose, maltose, sucrose and sugar are particularly preferably used. Either a single type or a plurality of types of these sweetness ingredients may be used.

### 3. Food or Beverage Containing Novel Steviol Glycoside

In one aspect of the present invention, a food or beverage containing the compound represented by Formula (1), or a derivative, a salt or a hydrate thereof (hereinafter, also referred to as the “food or beverage of the present invention”) is provided. The food or beverage of the present invention is not particularly limited as long as it contains the compound represented by Formula (1), or a derivative, a salt or a hydrate thereof, and it may be a food or beverage containing an extract containing the compound represented by Formula (1), or a derivative, a salt or a hydrate thereof. As used herein, a food or beverage refers to foods and beverages. Therefore, in some embodiments, the present invention provides a novel food or beverage, and a method for producing said food or beverage.

While the amount of the glycoside of the present invention contained in the food or beverage of the present invention differs depending on the specific food or beverage, it is preferably around 0.0004%-0.8% and particularly preferably 0.04%-0.4%. As long as the content lies within this range, the lingering aftertaste can advantageously be suppressed.

The food or beverage of the present invention may further contain other steviol glycosides. For example, the sweetener composition of the present invention may contain, in addition to the glycoside of the present invention, one or more types of steviol glycosides selected from the group consisting of rebaudioside A, rebaudioside B, rebaudioside C, rebaudioside D, rebaudioside E, rebaudioside F, rebaudioside I, rebaudioside J, rebaudioside K, rebaudioside N, rebaudioside M, rebaudioside O, rebaudioside Q, rebaudioside R, dulcoside A, rubusoside, steviol, steviol monoside, steviol bioside and stevioside.

In a case where other steviol glycoside is contained, the composition ratio of the glycoside of the present invention and other steviol glycoside is preferably 0.01:9.99-6:4 in a mass ratio.

The food or beverage of the present invention may further contain a general sweetener. Examples of such a general sweetener include natural sweeteners such as fructose, sugar, fructose-glucose syrup, glucose, maltose, sucrose, high-fructose syrup, sugar alcohol, oligosaccharide, honey, pressed sugarcane juice (brown sugar syrup), starch syrup, Lo Han Kuo (Siraitia grosvenorii) powder, Lo Han Kuo extract, licorice powder, licorice extract, Thaumatococcus daniellii seed powder and Thaumatococcus daniellii seed extract, and artificial sweeteners such as acesulfame potassium, sucralose, neotame, aspartame and saccharin. Among them, natural sweeteners are preferably used from the aspect of imparting clean taste, easy drinkability, natural flavor and moderately rich taste, where fructose, glucose, maltose, sucrose and sugar are particularly preferably used. Either a single type or a plurality of types of these sweetness ingredients may be used.

Examples of the food of the present invention include, but not particularly limited to, a confection, a bread, cereal flour, noodles, rice, a processed agricultural/forestry food, a processed livestock product, a processed fishery product, a milk dairy product, an oil-and-fat/processed oil-and-fat product, seasoning or other food material.

Examples of the beverage of the present invention include, but not particularly limited to, a carbonated beverage, a non-carbonated beverage, an alcoholic beverage, a non-alcoholic beverage, a coffee beverage, a tea beverage, a cocoa beverage, a nutritious beverage and a functional beverage.

The beverage of the present invention may be heat sterilized and packaged to be prepared as a packaged beverage. Examples of such package include, but not particularly limited to, a PET bottle, an aluminum can, a steel can, a paper package, a chilled cup, and a bottle. In a case where heat sterilization is to be performed, the type of heat sterilization is not particularly limited. For example, heat sterilization may be performed by employing a common technique such as UHT sterilization, retort sterilization or the like. While the temperature during the heat sterilization process is not particularly limited, it is, for example, 65-130° C., and preferably 85-120° C., for 10-40 minutes. Sterilization, however, can be carried out at an appropriate temperature for a several seconds, for example, 5-30 seconds, without problem as long as the same sterilizing value as that under the above-described conditions can be earned.

### 4. Plant Containing Novel Steviol Glycoside and Extract Thereof

In one aspect of the present invention, a plant containing the novel steviol glycoside and an extract thereof are provided. Furthermore, in another aspect of the present invention, a food or beverage, preferably a beverage, containing the plant of the present invention or an extract of the plant is provided. While the amount of the glycoside of the present invention contained in the plant of the present invention is not particularly limited, it is preferably 0.001%-1.000% and more preferably 0.01%-0.80%.

Preferably, the plant of the present invention is a plant that contains the glycoside of the present invention in a larger amount than a wild-type stevia species by 0.01% or more. As described above, the steviol glycoside of the present invention is not contained in a wild-type stevia at all or, if any, contained in an amount of the detection limit or less.

The phrase “contains the glycoside of the present invention in a larger amount than a wild-type stevia species by 0.01% or more” means that, with respect to an amount (concentration) of the glycoside of the present invention contained per unit quantity (e.g., 10 ml) of a liquid extract from fresh leaves (undried leaves) of a wild-type stevia plant, an amount (concentration) of the glycoside of the present invention contained in an equal unit quantity of a liquid extract from fresh leaves (undried leaves) of the plant of the present invention (the same amount as that of the liquid extract from the leaves of the wild-type stevia plant) is higher by 0.01% or more. Here, the plant of the present invention may contain the glycoside of the present invention in a larger amount than a wild-type stevia species by 0.02%6 or more, 0.03% or more, 0.04% or more, 0.05% or more, 0.07% or more, 0.09% or more, 0.10% or more, 0.15% or more, 0.20% or more, 0.40% or more, 0.60% or more, 0.80% or more, 1.0% or more, 1.50% or more, 2.00% or more, 4.00% or more, 6.00% or more, 8.00% or more, or 10.00% or more.

Moreover, the phrase “the proportion of the glycoside of the present invention among the total steviol glycosides is 0.01% or more” means that the glycoside of the present invention exists at a percentage of 0.01% or more with respect to the total content of the steviol glycosides existing in the liquid extract from the fresh leaves (undried leaves) of the stevia plant of the present invention. Here, the total steviol glycosides neither contain unknown steviol glycosides nor any steviol glycoside existing in an amount less than the detection limit. Preferably, the total steviol glycosides consist of rebaudioside A, rebaudioside B, rebaudioside C, rebaudioside D, rebaudioside E, rebaudioside F, rebaudioside I, rebaudioside J, rebaudioside K, rebaudioside N, rebaudioside M, rebaudioside O, rebaudioside Q, rebaudioside R, dulcoside A, rubusoside, steviol, steviol monoside, steviol bioside and stevioside.

While the content of the glycoside of the present invention in the plant of the present invention is as described above, in a case where dried leaves are obtained from the plant of the present invention, the glycoside of the present invention may exist in an amount of 0.01 wt % or more, 0.02 wt % or more, 0.03 wt % or more, 0.04 wt %/o or more, 0.05 wt % or more, 0.07 wt % or more, 0.10 wt % or more, 0.15 wt % or more, 0.20 wt % or more, 0.30 wt % or more, 0.50 wt % or more, 0.60 wt or more, 0.80 wt % or more, 1.00 wt % or more, 2.00 wt % or more, 4.00) wt % or more, 6.00 wt % or more, 8.00 wt % or more, or 10.00 wt % or more with respect to the weight of said dried leaves.

Here, dried leaves of the plant of the present invention refer to those obtained by drying fresh leaves of the plant of the present invention to reduce their water content to 10 wt % or less, 7 wt % or less, 5 wt % or less, 4 wt % or less, 3 wt %° or less, 2 wt % or less, or 1 wt % or less. Preferably, the water content of the dried leaves of the plant of the present invention is 3-4 wt %.

An example of the plant of the present invention include a plant with abundant Reb.C. As described above, the steviol glycoside of the present invention is not contained in a wild-type stevia or an extract thereof at all or, if any, contained in an amount of the detection limit or less. Meanwhile, the present inventors found out that the steviol glycoside of the present invention is contained in a larger amount in a plant having abundant Reb.C. Therefore, the novel steviol glycoside and the extract thereof also comprise such a plant with abundant Reb.C and an extract thereof.

An example of such a plant with abundant Reb.C include, but not particularly limited to, a high-rebaudioside C-containing non-recombinant stevia plant which contains rebaudioside C in a larger amount than a wild-type stevia species by 20% or more, and whose proportion of rebaudioside C among the total steviol glycosides is 40% or more (hereinafter, also referred to as a “high-Reb.C plant”).

An example of such a high-Reb.C plant include a high-rebaudioside C-containing non-recombinant stevia plant which contains rebaudioside C in a larger amount than a wild-type stevia species by 20% or more, and whose proportion of rebaudioside C among the total steviol glycosides is 40% or more.

A high-Reb.C plant is a cultiver derived from a plant of a wild-type stevia, in which genetic mutation has occurred to increase rebaudioside C. Examples of such genetic mutation include, but not particularly limited to, genetic mutation induced under naturally occurring conditions, genetic mutation induced by a non-recombinational technique and genetic mutation induced by genetic recombination.

A high-Reb.C plant can be screened, for example, by detecting gene polymorphism in the tissue of the plant. Herein. “screening” means to identify a high-Reb.C plant among other plants and to select the high-Reb.C plant.

The high-Reb.C plant may also be screened according to a screening method that includes a step of identifying a polymorphism of A in the wild type being altered to T at the 60th nucleotide of the nucleotide sequence represented by SEQ ID NO: 11 in the genome of a test plant.

The plant of the present invention not only comprises the whole plant but may also include plant organs (for example, leaf, petal, stem, root, seed, etc.), plant tissues (for example, epidermis, phloem, parenchyma, xylem, vascular bundles, palisade tissue, spongy tissue, etc.), various forms of plant cells (for example, suspension cultured cells), a protoplast, a leaf piece, callus and the like.

In addition, the plant of the present invention may also comprise a tissue culture or a plant cell culture. This is because such a tissue culture or plant cell culture may be cultured to regenerate a plant. Examples of the tissue culture or the plant cell culture of the plant of the present invention include, but not limited to, an embryo, meristematic cells, pollen, a leaf, a root, a root apex, a petal, a protoplast, a leaf piece and callus.

An extract of the plant of the present invention can be obtained by reacting a fresh leaf or a dried leaf of the plant of the present invention with an appropriate solvent (an aqueous solvent such as water or an organic solvent such as alcohol, ether or acetone). For conditions for extraction, see a method described in WO2016/090460 or a method described in the example below.

Preferably, the extract of the plant of the present invention contains the glycoside of the present invention in a larger amount than a wild-type stevia by 0.01% or more, where the proportion of the glycoside of the present invention among the total steviol glycosides is 0.01% or more. Here, the phrase “contains the glycoside of the present invention in a larger amount than a wild-type stevia by 0.01% or more” means the same as described above. Similarly, the phrase the “proportion of the glycoside of the present invention among the total steviol glycosides is 0.01% or more” also means the same as described above.

### 5. Flavor Controlling Agent Containing Novel Steviol Glycoside

Although the novel steviol glycoside of the present invention is contained in a stevia extract in a minute quantity, it is considered to have an influence on the flavor of the stevia extract. While not wishing to be bound by any theory, addition of a small amount of the steviol glycoside of the present invention is presumably capable of controlling the flavor of a food or beverage. Therefore, in one aspect of the present invention, a flavor controlling agent containing the above-described compound represented by Formula (1) or a derivative, a salt or a hydrate thereof is provided.

As used herein, a “flavor controlling agent” refers to a substance that can be added to a food or beverage to control the flavor of the food or beverage. Preferably, the flavor controlling agent of the present invention can be added to a food or beverage so as to control the flavor of the food or beverage itself without the consumers recognizing the taste of the flavor controlling agent itself. For example, since the steviol glycoside of the present invention has good lingering sweet aftertaste as compared to conventional steviol glycosides, it can be used as a flavor controlling agent for controlling the lingering sweet aftertaste of the food or beverage.

The flavor controlling (enhancing) agent of the present invention preferably contains, in addition to the above-described compound represented by Formula (1) or a derivative, a salt or a hydrate thereof, one or more types of other sweeteners. Examples of such sweetener include: one or more types of steviol glycosides selected from the group consisting of rebaudioside A, rebaudioside D, rebaudioside B, rebaudioside M, rebaudioside N, rebaudioside O, rebaudioside E, rebaudioside K and rebaudioside J; natural sweeteners such as fructose, sugar, fructose-glucose syrup, glucose, maltose, sucrose, high-fructose syrup, sugar alcohol, oligosaccharide, honey, pressed sugarcane juice (brown sugar syrup), starch syrup, Lo Han Kuo (Siraitia grosvenorii) powder, Lo Han Kuo extract, licorice powder, licorice extract, Thaumatococcus daniellii seed powder and Thaumatococcus daniellii seed extract; and artificial sweeteners such as acesulfame potassium, sucralose, neotame, aspartame and saccharin.

In one aspect of the present invention, the flavor controlling agent of the present invention is a flavor controlling agent that improves lingering aftertaste and that contains the compound represented by Formula (1) or a derivative, a salt or a hydrate thereof. While Brix of commercially available foods or beverages (for example, refreshing beverages) is usually up to around 15, reduction in the amount of sugar in foods or beverages has been considered due to the recent growth of health consciousness and the introduction of sugar tax. Where the amount of sugar is reduced, use of a non-sugar sweetener (for example, a non-calorie sweetener) has been attempted to compensate for the loss of Brix due to the sugar reduction. For example, if the amount of sugar in a food or beverage originally having Brix of 10 is reduced by half, Brix will be 5. Therefore, there is a need to add a non-sugar sweetener to make up for the sweetness level corresponding to Brix of 5. Many of the non-sugar sweeteners, however, have unique flavors that differ from sugar, where one of such characteristic flavors is bad lingering sweet aftertaste. Since the steviol glycoside of the present invention has good lingering sweet aftertaste, a flavor controlling agent containing the steviol glycoside of the present invention can be used as a flavor controlling agent for improving the lingering aftertaste. Herein, “Brix” is a scale of a sweetness level of a food or beverage, that is, a value of the concentration of the soluble solid content expressed in a weight percent concentration in a sucrose solution at 20° C. Accordingly, it is represented by an amount of sucrose (g) in 100 g of an aqueous sucrose solution. For example, Brix of 5 represents a sweetness level corresponding to the sweetness level of 5 g of sucrose in 100 g of an aqueous sucrose solution.

The flavor controlling agent of the present invention is preferably added to the non-sugar sweetener contained in a food or beverage in an amount of 1 mass %-15 mass % based on the mass of the sweetener. The flavor controlling agent of the present invention is added more preferably in an amount of 1.5 mass %-12 mass %, and still more preferably in an amount of 3.5 mass %-11 mass % based on the mass of the sweetener. The content of the non-sugar sweetener in the food or beverage added with the flavor controlling agent of the present invention is preferably 5-13, more preferably 5-12 and still more preferably 5-7 in terms of Brix. Here, the phrase “the content of the non-sugar sweetener is 5 in terms of Brix” refers to a content that gives the sweetness of the aqueous solution containing the non-sugar sweetener to be equivalent to Brix of 5. For example, if the sweetness level of a non-sugar sweetener is sweeter than sugar by 200 times, the amount that gives “the content of the non-sugar sweetener to be 5 in terms of Brix” is 0.025 g in 100 g of an aqueous solution containing the non-sugar sweetener. In a preferable aspect of the present invention, a flavor controlling agent of the present invention is added to the non-sugar sweetener contained in the food or beverage in an amount of 1 mass %-15 mass % based on the mass of the sweetener, wherein the content of the non-sugar sweetener is 5.5-12 in terms of Brix. In other preferable aspect of the present invention, the flavor controlling agent of the present invention is added to the non-sugar sweetener contained in the food or beverage in an amount of 1.5 mass %-12 mass % based on the mass of the sweetener, wherein the content of the non-sugar sweetener is 5-13 in terms of Brix.

Examples of other sweetener contained in the food or beverage include, but not limited to: one or more types of steviol glycoside selected from the group consisting of rebaudioside A, rebaudioside D, rebaudioside B, rebaudioside M, rebaudioside N, rebaudioside O, rebaudioside E, rebaudioside K and rebaudioside J; natural sweeteners such as fructose, fructose-glucose syrup, glucose, maltose, high-fructose syrup, sugar alcohol, oligosaccharide, honey, pressed sugarcane juice (brown sugar syrup), starch syrup, Lo Han Kuo (Siraitia grosvenorii) powder, Lo Han Kuo extract, licorice powder, licorice extract, Thaumatococcus daniellii seed powder and Thaumatococcus daniellii seed extract; and artificial sweeteners such as acesulfame potassium, sucralose, neotame, aspartame and saccharin.

In another aspect of the present invention, a flavor controlling agent of the present invention is a flavor controlling agent containing the above-described compound represented by Formula (1) or a derivative, a salt or a hydrate thereof for enhancing sweetness. A flavor controlling agent for enhancing sweetness refers to a flavor controlling agent that can be added to a food or beverage containing a sweetener so that it can impart stronger sweetness to the food or beverage than a simple sum of the sweetness of the flavor controlling agent thereto. For example, when a flavor controlling agent of an amount equivalent to Brix of 0.1 is added to a food or beverage with sweetness equivalent to Brix of 9, the flavor controlling agent should be capable of imparting a sweetness level exceeding Brix of 9.1 (for example, Brix of 9.2) to the food or beverage. Use of such a flavor controlling agent for enhancing sweetness can reduce the total amount of the sweeteners used, and thus advantageous in realizing calorie reduction and cost reduction.

If the sweetness level of the sweetener targeted for sweetness enhancement is 1-10, the flavor controlling agent for enhancing sweetness of the present invention is preferably added in an amount of 0.05 mass %-2.0 mass %, more preferably added in an amount of 0.1 mass %-1.5 mass %, and still more preferably added in an amount of 0.2 mass %-1.2 mass % based on the mass of the sweetener targeted for sweetness enhancement.

Examples of the sweetener targeted for sweetness enhancement include, but not particularly limited to: one or more types of steviol glycoside selected from the group consisting of rebaudioside A, rebaudioside D, rebaudioside B, rebaudioside M, rebaudioside N, rebaudioside O, rebaudioside E, rebaudioside K and rebaudioside J; natural sweeteners such as fructose, sugar, fructose-glucose syrup, glucose, maltose, sucrose, high-fructose syrup, sugar alcohol, oligosaccharide, honey, pressed sugarcane juice (brown sugar syrup), starch syrup, Lo Han Kuo (Siraitia grosvenorii) powder, Lo Han Kuo extract, licorice powder, licorice extract, Thaumatococcus daniellii seed powder and Thaumatococcus daniellii seed extract; and artificial sweeteners such as acesulfame potassium, sucralose, neotame, aspartame and saccharin.

### 6. Method for Producing Novel Steviol Glycoside

As described above, the steviol glycoside of the present invention can be produced by (A) isolation/purification from a plant, (B) a chemical synthesis, or (C) a biosynthesis. Hereinafter, each of them will be described.

(A) Isolation/Purification from Plant

Since the plant of the present invention contains the novel steviol glycoside of the present invention, the novel steviol glycoside can be isolated/purified from said plant. A fresh or dried leaf of the plant of the present invention is allowed to react with an appropriate solvent (an aqueous solvent such as water or an organic solvent such as alcohol, ether or acetone) to extract the novel steviol glycoside in a liquid extract state. For extraction conditions and else, see the method described in WO2016/090460 or the method described in the example below.

Furthermore, the resulting liquid extract may be subjected to a known method such as a gradient of ethyl acetate or other organic solvent: water, high performance liquid chromatography (HPLC), or ultra (high) performance liquid chromatography (UPLC) to isolate/purify the novel steviol glycoside.

The content of the novel steviol glycoside in the plant can be determined by the method described in WO2016/090460 or the method described in the example below. Specifically, the content can be measured by sampling fresh leaves from the plant of the present invention and subjecting the leaves to LC-MS/MS.

### (2) Chemical Synthesis

A method for synthesizing the steviol glycoside of the present invention will be described in detail hereinbelow.

Steviol glycosides have structures in which different sugar moieties (glucose, rhamnose, xylose, etc.) are attached to the aglycone, i.e., steviol, via various linkage forms (linkage positions and conformations). Therefore, a steviol glycoside of interest can be obtained via various pathways depending on the selected starting material. Those skilled in the art to which the present invention pertains, however, wound understand that the time and the yield for obtaining the compound of interest greatly vary depending on the synthetic pathways.

This time, the present inventors found out a novel method for producing a steviol glycoside of the present invention with higher selectivity and higher yield via a specific synthetic pathway. According to the method for synthesizing the steviol glycoside of the present invention, a chemical synthesis of the steviol glycoside proceeds by separating the steviol glycoside into a “steviol glycoside” and a “sugar hemiacetal form” as shown in Scheme 1.

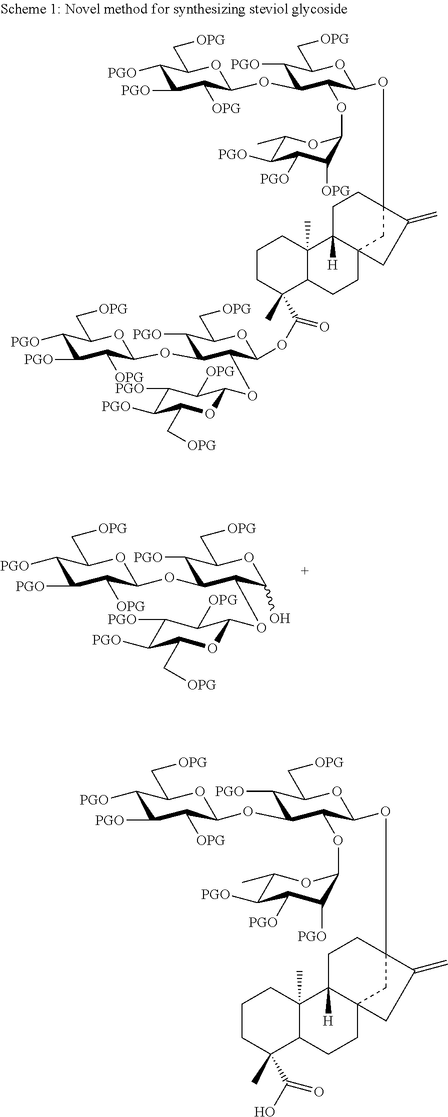

The steviol glycoside can be prepared by deriving from an existing natural substance (rebaudioside, dulcoside, stevioside, steviol bioside, rubusoside, etc.). Meanwhile, the sugar hemiacetal form can be prepared either from an existing natural substance or by a chemical synthesis. The present inventors found that the steviol glycoside of interest can be obtained with good yield and extremely high n-selectivity by condensing the steviol glycoside and the sugar hemiacetal form through the Mitsunobu reaction.

In one aspect of the present invention, a method for producing the compound represented by Formula (1) is provided, where the method comprises the steps of:

(A) synthesizing a compound represented by Formula (3) below:

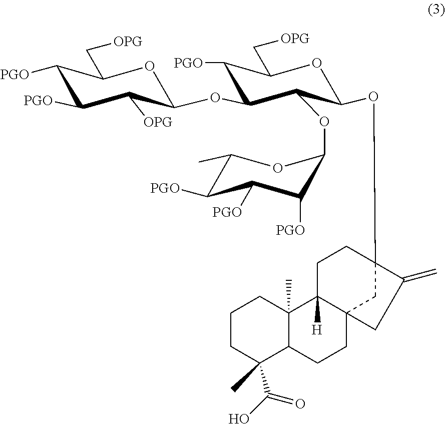

(wherein, PGs each independently represents a protecting group)
from rebaudioside C represented by Formula (2) below:

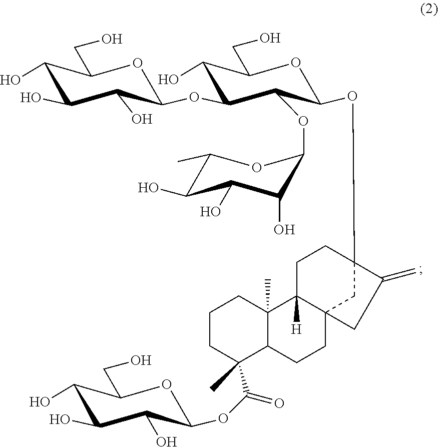

and

(B) synthesizing a compound represented by Formula (4) below

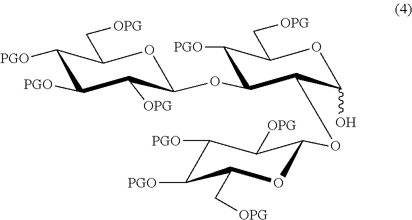

(wherein, PGs each independently represents a protecting group)
from a glucopyranoside derivative.

In another aspect of the present invention, the method for producing the compound represented by Formula (1) is provided, where the method further comprises a step of allowing reaction between the compound represented by Formula (3) above and the compound represented by Formula (4) above in the presence of a phosphine reagent and an azo compound to obtain a compound represented by Formula (5) below

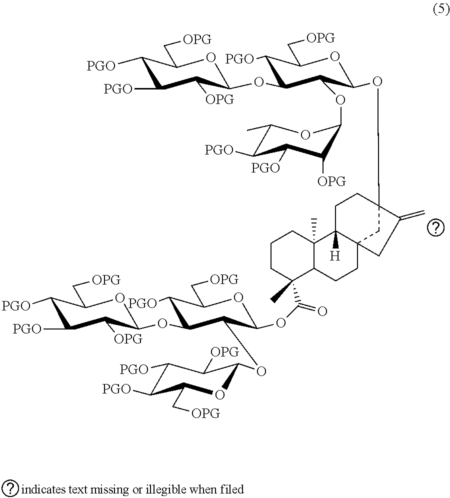

(wherein, PGs each independently represents a protecting group).

Herein, examples of the protecting group include an acyl protecting group, a trisubstituted silyl group, an acetal protecting group and an ether protecting group. Preferable examples include a trisubstituted silyl group (a trimethylsilyl group, a triethylsilyl group, a t-butyldimethylsilyl group, etc.) and an acyl protecting group (an acetyl group, a benzoyl group, etc.).

(A) First Step (Synthesis of Steviol Glycoside)

A steviol glycoside can be obtained, for example, by following Scheme 2 below using naturally occurring rebaudioside C (dulcoside B) as a raw material.

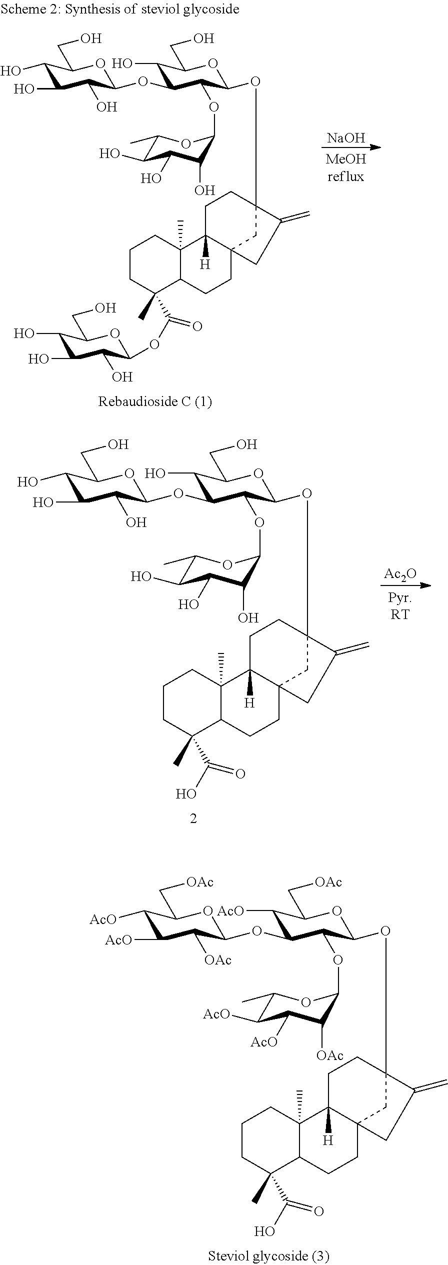

First, rebaudioside C is dissolved in a solvent such as methanol and water, added with a strong base such as sodium hydroxide, and refluxed at 60° C.-120° C. for 2 hours or longer so that the glucose molecule is removed from C-19 of rebaudioside C to give Compound 2 above. In doing so, the solvent may be evaporated after neutralizing the reaction solution with a cation exchange resin or the like.

Compound 2 is further dissolved in a solvent such as pyridine, and added with acetic anhydride or the like to protect the hydroxyl groups contained in Compound 2, thereby obtaining Compound 3.

(B) Second Step (Synthesis of Trisaccharide Hemiacetal)

The trisaccharide hemiacetal can be obtained, for example, by following Scheme 3 below using a commercially available glucopyranoside derivative as a raw material.

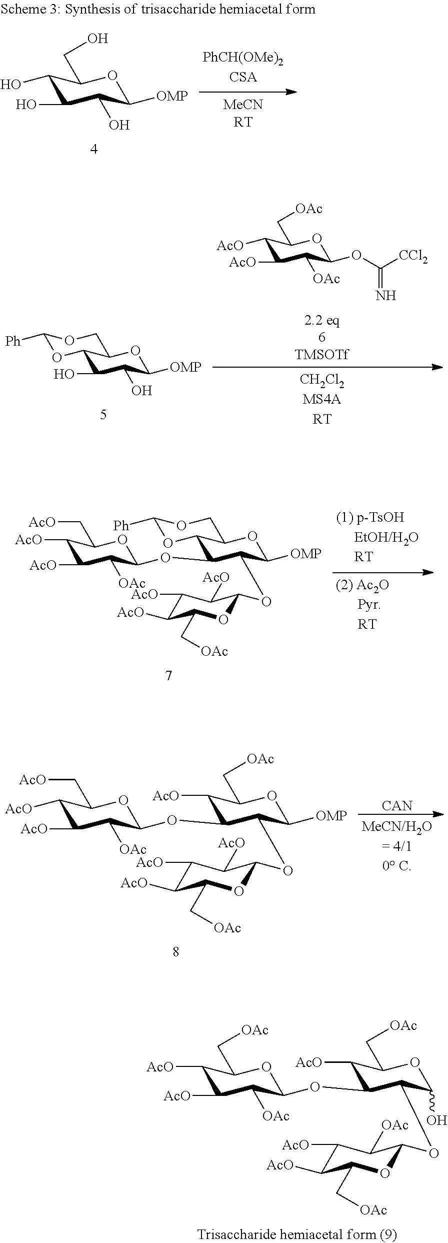

First, 4-methoxyphenyl β-D-glucopyranoside (4) is dissolved in a solvent such as acetonitrile, added with benzaldehyde dimethyl acetal and camphorsulfonic acid (acid catalyst), and agitated at 25° C.-80° C. for 2 hours or longer to give Compound 5. Subsequently, Compound 5, 2,3,4,6-tetra-O-acetyl-β-D-glucosypyranosyl 2,2,2-trichloroacetimidate (6) and 4 Å molecular sieves are dissolved in a solvent such as dichloromethane, added with trimethylylsilyl trifluoromethanesulfonate at a low temperature (e.g., 0° C.), and agitated at room temperature for 2 hours or longer to give Compound 7.

Compound 7 is dissolved in a solvent such as ethanol, added with P-toluenesulfonic acid at room temperature, agitated at 60° C.-80° C. for 2 hours or longer to complete the reaction, then neutralized with triethylamine, and concentrated under a reduced pressure. The resulting syrup is dissolved in a solvent such as pyridine, and added with acetic anhydride or the like to give Compound 8 with protected hydroxyl groups. Compound 8 is dissolved in acetonitrile and water, added with an oxidant such as cerium ammonium nitrate, and agitated for 5 minutes to 2 hours, thereby obtaining Compound 9.

(C) Third Step (Synthesis of Compound Represented by Formula (1))

The compound represented by Formula (1) can be synthesized, for example, by following Scheme 4 below using Compounds 3 and 9 obtained in Steps 1 and 2 above.

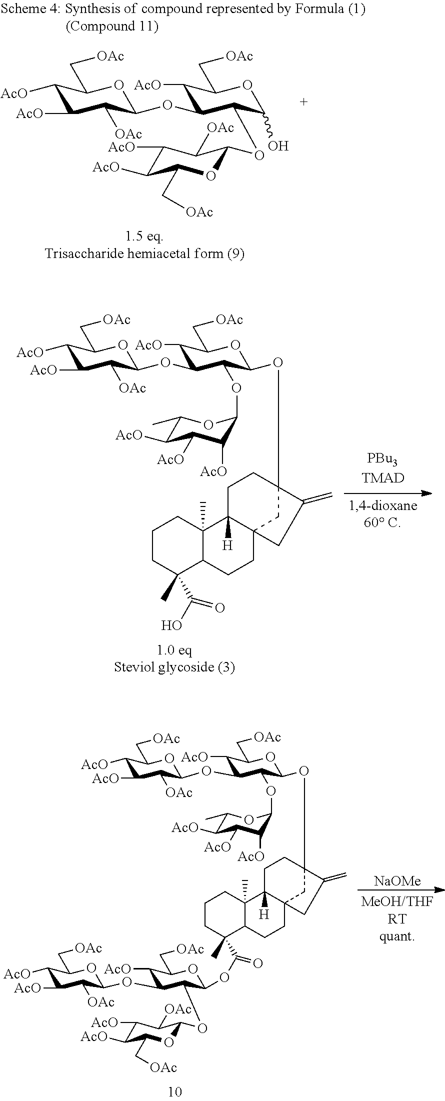

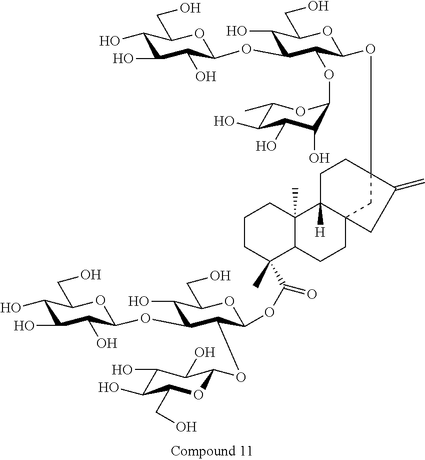

First, the trisaccharide hemiacetal form and the steviol glycoside obtained in Steps 1 and 2 are allowed to undergo the Mitsunobu reaction so that only Compound 10 in the β-form can selectively be obtained with very high yield (45% or more). Specifically, these compounds are dissolved in 1,4-dioxane, added with a phosphine reagent such as tributylphosphine or triphenylphosphine and an azo compound such as 1,1′-azobis (N,N′-dimethylformamide) (TMAD) at room temperature, and agitated at 50° C.-80° C. for 2 hours or longer to give only Compound 10 in the β-form. Finally, the protecting groups of Compound 10 are deprotected to give the compound represented by Formula (1) (Compound 11).

(3) Biosynthesis

The steviol glycoside of the present invention can also be generated by transferring a polynucleotide coding for a predetermined protein into a host cell derived from a bacterium, a plant, an insect, a non-human mammal or the like, and using steviol, a steviol glycoside, UDP-glucose and/or UDP-rhamnose as a substrate. Steviol, a steviol glycoside, UDP-glucose or UDP-rhamnose as the substrate may be either provided or biosynthesized in the cell. While examples of the predetermined protein include stevia-derived UGT85C2 (the amino acid sequence represented by SEQ ID NO:2), UGT74G1 (the amino acid sequence represented by SEQ ID NO:4), UGT91D2 (the amino acid sequence represented by SEQ ID NO:6), UGT76G1 (the amino acid sequence represented by SEQ ID NO:8) and Arabidopsis thaliana-derived UDP-rhamnose synthase AtRHM2 (the amino acid sequence represented by SEQ ID NO: 10), it is not limited thereto as long as it has an equivalent activity.

The above-described protein is an enzyme derived from Arabidopsis thaliana or stevia, which is expected to be highly active in an environment outside plant cells such as Arabidopsis thaliana and stevia (for example, in an extracellular environment, or inside a host cell other than stevia). In this case, the polynucleotide coding for the above-described protein (for example, UGT85C2 gene is represented by SEQ ID NO: 1, UGT74G1 gene is represented by SEQ ID NO:3, UGT91 D2 gene is represented by SEQ ID NO:5, UGT76G1 gene is represented by SEQ ID NO:7 and AtRHM2 gene is represented by SEQ ID NO:9) is transferred into a host cell derived from a bacterium, a fungus, a plant, an insect or a non-human mammal so as to allow expression of the protein of the present invention, to which steviol, a steviol glycoside, UDP-glucose or UDP-rhamnose as the substrate is provided to generate the compound of the present invention. Alternatively, depending in the host, the above-described protein is expressed in the host cell, to which an appropriate substrate is provided to generate the compound of the present invention.

In one aspect of the present invention, a method for producing the novel steviol glycoside of the present invention is provided, where the method is characterized by use of a non-human transformant that has been introduced with at least one of polynucleotides (a) to (g) below.

(a) A polynucleotide containing the nucleotide sequence of SEQ ID NO: 1, a polynucleotide containing a nucleotide sequence having 90% or higher identity with the nucleotide sequence of SEQ ID NO: 1, or a polynucleotide coding for a protein that has 90% or higher identity with the amino acid sequence of SEQ ID NO:2 and that has an activity of adding glucose to the hydroxyl group at C-13 of the steviol glycoside.

(b) A polynucleotide containing the nucleotide sequence of SEQ ID NO:3, a polynucleotide containing a nucleotide sequence having 90% or higher identity with the nucleotide sequence of SEQ ID NO:3, or a polynucleotide coding for a protein that has 90% or higher identity with the amino acid sequence of SEQ ID NO:4 and that has an activity of adding glucose to the carboxylic acid at C-19 of the steviol glycoside.

(c) A polynucleotide containing the nucleotide sequence of SEQ ID NO:5, a polynucleotide containing a nucleotide sequence having 90% or higher identity with the nucleotide sequence of SEQ ID NO:5, or a polynucleotide coding for a protein that has 90% or higher identity with the amino acid sequence of SEQ ID NO:6 and that has an activity of adding rhamnose to glucose attached to C-13 of the steviol glycoside via a 1→2 linkage.

(d) A polynucleotide containing the nucleotide sequence of SEQ ID NO: 7, a polynucleotide containing a nucleotide sequence having 90% or higher identity with the nucleotide sequence of SEQ ID NO:7, or a polynucleotide coding for a protein that has 90% or higher identity with the amino acid sequence of SEQ ID NO:8 and that has an activity of adding glucose to C-3 of glucose at C-13 of the steviol glycoside via a 1→3 linkage.

(e) A polynucleotide containing the nucleotide sequence of SEQ ID NO:5, a polynucleotide containing a nucleotide sequence having 90% or higher identity with the nucleotide sequence of SEQ ID NO:5, or a polynucleotide coding for a protein that has 90% or higher identity with the amino acid sequence of SEQ ID NO:6 and that has an activity of adding glucose to glucose at C-19 of the steviol glycoside via a 1→2 linkage.

(f) A polynucleotide containing the nucleotide sequence of SEQ ID NO:7, a polynucleotide containing a nucleotide sequence having 90% or higher identity with the nucleotide sequence of SEQ ID NO:7, or a polynucleotide coding for a protein that has 90% or higher identity with the amino acid sequence of SEQ ID NO:8 and that has an activity of adding glucose to glucose at C-19 of the steviol glycoside via a 1→3 linkage.

(g) A polynucleotide containing the nucleotide sequence of SEQ ID NO:9, a polynucleotide containing a nucleotide sequence having 90% or higher identity with the nucleotide sequence of SEQ ID NO:9, or a polynucleotide coding for a protein that has 90% or higher identity with the amino acid sequence of SEQ ID NO: 10 and that has an activity of generating UDP-rhamnose from UDP-glucose.

In a preferable aspect of the present invention, polynucleotides independently having 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8%0/or higher, or 99.9% or higher sequence identity with the nucleotide sequences of the sequence numbers mentioned in (a) to (g) above can be used.

In another preferable aspect of the present invention, proteins that independently have an amino acid sequence having 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher sequence identity with the amino acid sequences of the sequence number mentioned in (a) to (g) above and that has the predetermined activity described in (a) to (g) above can be used.

Preferably, a polynucleotide coding for the above-described protein is introduced into a host while being inserted into an appropriate expression vector. The polynucleotides may individually be inserted into separate vectors.

An appropriate expression vector is generally made to contain:

(i) a promoter that allows transcription in the host cell:

(ii) a polynucleotide of the present invention linked to said promoter; and

(iii) an expression cassette that is involved in transcription termination and polyadenylation of RNA molecules and that contains, as a component thereof, a signal that functions in the host cell.

Examples of a method for preparing an expression vector include, but not particularly limited to, a method that uses a plasmid, a phage, a cosmid or the like, and DNA molecules having necessary components.

The type of the vector is not particularly limited, and any vector that allows expression in the host cell can suitably be selected. Specifically, a promoter sequence is suitably selected according to the type of the host cell to ensure expression of the polynucleotide of the present invention, and a vector obtained by integrating this promoter sequence and the polynucleotide of the present invention into a plasmid or the like is used as an expression vector.

The expression vector of the present invention includes expression controlling regions (for example, a promoter, a terminator and/or an origin of replication and the like) depending on the type of the host into which it is introduced. A promoter used in a bacterial expression vector may be a common promoter (for example, a trc promoter, a tac promoter, a lac promoter, etc.), a promoter used for a yeast may be, for example, a glyceraldehyde-3-phosphate dehydrogenase promoter, PH05 promoter, a GAL 1/10 promoter or the like, and a promoter for filamentous fungi may be, for example, amylase, trpC or the like. Moreover, examples of a promoter for expressing the gene of interest in a plant cell include a cauliflower mosaic virus 35S RNA promoter, a rd29A gene promoter, a rbcS promoter, and a mac-1 promoter in which the enhancer sequence of the cauliflower mosaic virus 35S RNA promoter is provided at the 5′ end of a promoter sequence of Agrobacterium-derived mannopine synthase. A promoter for an animal cell host may be a viral promoter (for example, a SV40 early promoter, a SV40 late promoter, etc.). Examples of a promoter that is inducibly activated in response to external stimuli include a mouse mammary tumor virus (MMTV) promoter, a tetracycline responsive promoter, a metallothionein promoter and a heat shock protein promoter.

Preferably, the expression vector contains at least one selectable marker. As such a marker, an auxotrophic marker (LEU2, URA3, HIS3, TRP 1, ura5, niaD), a drug resistance marker (hygromycin, zeocin), a geneticin resistance gene (G418r), a copper resistance gene (CUPI) (Marin et al., Proc. Natl. Acad. Sci. USA, vol. 81, p. 337, 1984), a cerulenin resistance gene (fas2m, PDR4) (Junji Inokoshi et al., Journal of Japanese Biochemical Society, vol. 64, p. 660, 1992: Hussain et al., Gene, vol. 101, p. 149, 1991, respectively) or the like can be used.

As a method for transforming a host cell, a generally employed known method can be employed. For example, an electroporation method (Mackenxie, D. A. et al., Appl. Environ. Microbiol., vol. 66, p. 4655-4661, 2000), a particle delivery method (Japanese Unexamined Patent Application Publication No. 2005-287403), a spheroplast method (Proc. Natl. Acad. Sci. USA, vol. 75, p. 1929, 1978), a lithium acetate method (J. Bacteriology, vol. 153, p. 163, 1983), a method described in Methods in yeast genetics, 2000 Edition: A Cold Spring Harbor Laboratory Course Manual, or the like can be performed although the present invention is not limited thereto.

In addition, as to general molecular biological processes, see “Sambrook and Russell, Molecular Cloning: A Laboratory Manual Vol. 3. Cold Spring Harbor Laboratory Press 2001”, “Methods in Yeast Genetics, A laboratory manual (Cold Spring Harbor Laboratory Press, Cold Spring Harbor, N.Y.)” and the like.

A non-human transformant obtained as described above can be cultured so as to allow the non-human transformant to produce a steviol glycoside. Such a non-human transformant is preferably a yeast. Moreover, the non-human transformant is preferably cultured in a medium containing steviol. The accumulated steviol glycoside can be extracted/purified to obtain the steviol glycoside of the present invention.

### Example

[Isolation of Novel Steviol Glycoside]

Extracts obtained from the leaves of four lines of novel stevia plants (Sample 1 (EM3-4), Sample 2 (EM2-27-8), Sample 3 (EM2-27-15) and Sample 4 (EM2-11)) developed at Suntory Global Innovation Center (SIC) were subjected to high performance liquid chromatography (HPLC)-mass spectrometry (MS) for the screening analysis of the contained steviol glycoside based on the molecular weights of a steviol glycoside that had a sugar chain formed of D-glucopyranosyl (glc), L-rhamnopyranosyl (rha) and xylopyranosyl (xyl). Here, Sample 1 is a high-Reb.C plant having a genome polymorphism of A in the wild type being altered to T at the 60th nucleotide of the nucleotide sequence represented by SEQ ID NO: 11 in the genome of a test plant. A statistical analysis of the correlation between the phenotype having a high-RebC concentration and the polymorphism of SEQ ID NO: 11 revealed that said polymorphism had a statistic correlation with the phenotype having a high-RebC concentration.

A process for preparing a test liquid was as follows: 10.0 mg each of lyophilized dried stevia leaves was weighed into a glass vial, to which 1.0 mL of water/methanol (1/1 vol/vol) was added as an extracting solvent, and then the resultant was subjected to ultrasonic irradiation in an ultrasonic cleaner (AS ONE, AS52GTU) at a set temperature of 25° C. for 20 minutes, thereby obtaining a liquid extract of a steviol glycoside from the stevia leaves. The resultant was further 10-fold diluted with water/methanol and filtrated through a filter with a pore size of 0.45 μm (Nacalai tesque, Cosmonice filter S (solvent)) before being subjected to HPLC-MS.

For the HPLC part of HPLC-MS, Nexera LC-30AD (Shimadzu Corporation) was used as a liquid delivery unit LC pump, and SM-C18 (4.6×250 mm) (from Imtakt) as a separation column. Liquid delivery of the LC mobile phase was carried out by using 0.2% acetic acid-containing Milli-Q water as mobile phase A and methanol as mobile phase B, where the binary gradient was such that the concentration of the mobile phase B was constantly maintained at 10% for 0-5 minutes, the concentration of the mobile phase B was shifted from 10% to 70% in the next 15 minutes, then from 70% to 100% in the following 5 minutes, and finally ended by maintaining the concentration of the mobile phase B at 100% for 5 minutes. The flow rate of the mobile phase was 0.4 mL/min, and the stevia leaf liquid extract diluted and filtrated with a filter was injected for 5 μL.

For the MS part, triple quadrupole mass spectrometer LCMS-8030 (Shimadzu Corporation) equipped with an electrospray ionization (ESI) ion source was used. The mass spectrometry measurement was carried out in a selected ion monitoring (SIM) mode by selecting the negative ion measurement mode and the m/z values. The m/z values were selected by calculation based on the molecular weights of a steviol glycoside that had a sugar chain formed of D-glucopyranosyl (glc), L-rhamnopyranosyl (rha) and xylopyranosyl (xyl). Accordingly, m/z=641.2 (glc (2)), 773.2 (glc (2), xyl (1)), 787.2 (glc (2), rha (1)), 803.2 (glc (3)), 935.3 (glc (3), xyl (1)), 949.3 (glc (3), rha (1)), 965.3 (glc (4)), 1095.4 (glc (3), rha (2)), 1097.4 (glc (4), xyl (1)), 1111.4 (glc (4), rha (1)), 1127.4 (glc (5)), 1257.5 (glc (4), rha (2)), 1259.5 (glc (5), xyl (1)), 1273.5 (glc (5), rha (1)), 1289.5 (glc (6)), 1435.6 (glc (6), rha (1)) were selected. Furthermore, an available high purity reagent, rebaudiosides A, B, D, F, M, N and O, stevioside, and dulcosides A and B were also measured under the same conditions so as to confirm the negative ion m/z values and the retention time of HPLC. The peak areas (arbitrary unit) of the mainly detected steviol glycosides are shown in Table 1.

TABLE 1
Peak areas (arbitrary unit) observed by STM measurement in HPLC-MS

| | Compound name | | | | | |
|---|---|---|---|---|---|---|
| | | | | | Compound | |
| | | | | | represented by | |
| | Rebaudioside A | Rebaudioside C | Rebaudioside D | Rebaudioside M | Formula (1) | Rebaudioside N |
| Retention | 29.60 | 29.96 | 28.00 | 28.66 | 27.70 | 28.18 |
| time (min) | | | | | | |
| Peak area | 29,669,582 | 30,122,062 | 1,428,384 | 1,030,603 | 140,947 | 772,570 |
| (Sample 1) | 46.97% | 47.69% | 2.26% | 1.63% | 0.22% | 1.22% |
| Peak area | 23,762,676 | 24,201,473 | 2,253,735 | 1,029,837 | 97,388 | 1,211,504 |
| (Sample 2) | 45.21% | 46.05% | 4.29% | 1.96% | 0.19% | 2.31% |
| Peak area | 15,386,726 | 5,872,656 | 3,585,775 | 3,296,579 | 89,988 | 896.549 |
| (Sample 3) | 52.82% | 20.16% | 12.31% | 11.32% | 0.31% | 3.08% |
| Peak area | 16,070,017 | 10,339,094 | 1,404,429 | 74,413 | 0 | 308,709 |
| (Sample 4) | 56.99% | 36.67% | 4.98% | 0.26% | 0.00% | 1.09% |

Two peaks were observed in the selected ion chromatogram of the steviol glycoside (m/z 1273.5) in which the modified sugar chain contained five glucose moieties (glc) and one rhamnose moiety (rha). The selected ion chromatogram of Sample 1(EM3-4) at m/z of 1273.5 is shown in FIG. 1.

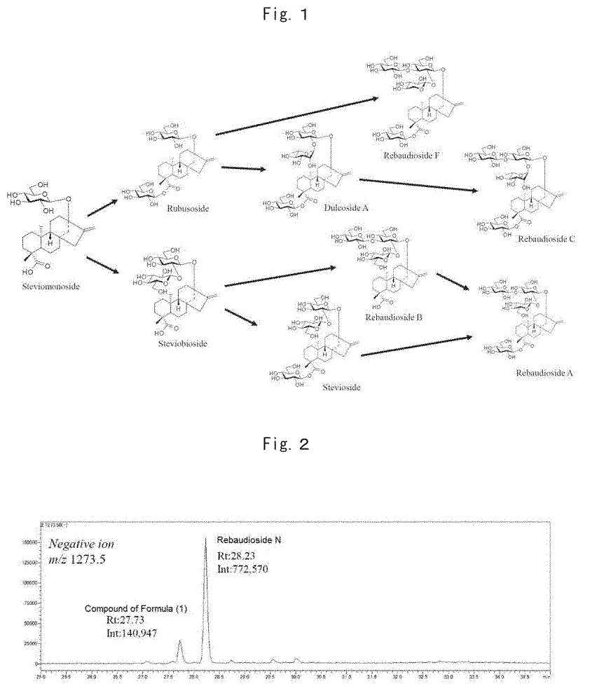

Of the two peaks shown in FIG. 2, the peak seen at the retention time (Rt) of 28.23 minutes matches the standard sample of rebaudioside N in terms of the mass value and the retention time. Meanwhile, no steviol glycoside has yet been reported to have a mass equivalent to that of rebaudioside N. Accordingly, the peak at Rt 27.73 minutes of the two peaks shown in FIG. 2 was found to be an unknown substance. For Sample 4 whose rebaudioside C content was lower than the content of rebaudioside A and whose sugar chain elongation was shorter than other samples, the peak value at Rt 27.73 was lower than the detection limit.

[Structural Analysis of Novel Steviol Glycoside]

According to the present invention, a structural analysis of the novel steviol glycoside detected in a cultiver with high rebaudioside C content was performed as follows.

(i) Structural deduction by a fragmentation analysis through high performance liquid chromatography (HPLC)-high resolution mass spectrometry (MS) and MS/MS, and three-stage ion fragmentation (MS³ fragmentation).

(ii) Chemical synthesis of the deduced steviol glycoside standard product via chemical reaction.

(iii) Structural confirmation by matching with the chemically synthesized standard product with respect to the retention time and the fragmented pattern from HPLC-high resolution MS and MS³ fragmentation

Hereinafter, each of Steps (i)-(iii) above will be described in detail.

(i) Structural deduction by a fragmentation analysis through high performance liquid chromatography (HPLC)-high resolution mass spectrometry (MS) and MS/MS, and three-stage ion fragmentation (MS³ fragmentation)

A process for preparing test liquids were as follows: 10.0 mg each of lyophilized dried stevia leaves was weighed into a glass vial, to which 1.0 mL of water/methanol (1/1 vol/vol) was added as an extracting solvent, and then the resultant was subjected to ultrasonic irradiation in an ultrasonic cleaner (AS ONE, AS52GTU) at a set temperature of 25° C. for 20 minutes, thereby obtaining the liquid extract of a steviol glycoside from the stevia leaves. The resultant was further 10-fold diluted with water/methanol and filtrated through a filter with a pore size of 0.45 μm (Nacalai tesque, Cosmonice filter S (solvent)) before being subjected to HPLC-MS.

In an equipment configuration for high performance liquid chromatography-electrospray ionization-accurate mass spectrometry (HPLC-ESI-HRMS), equipment for HPLC was configured by using Prominence LC-20AD (Shimadzu Corporation) as a liquid delivery unit LC pump and SM-C18 (4.6×250 mm) (from Imtakt) as a separation column. The LC mobile phase was delivered using 0.2% acetic acid-containing Milli-Q water as mobile phase A and methanol as mobile phase B, where the binary gradient was such that the concentration of the mobile phase B was constantly maintained at 10% for 0-5 minutes, the concentration of the mobile phase B was shifted from 10% to 70% in the next 15 minutes, and further from 70% to 100% in the following 5 minutes. Finally, the concentration of the mobile phase B was maintained at 100% for 5 minutes to end. The flow rate of the mobile phase was 0.4 mL/min, and the stevia leaf liquid extract diluted and subsequently filtrated with a filter was injected for 5 μL. For the mass spectrometry part, Orbitrap Elite MS (from Thermo Fisher Scientific) equipped with an ESI ion source was used. The mass spectrometry measurement was carried out in a negative ion measurement mode at m/z in a range of 150-2000 with resolution set to 60,000. The MS/MS measurement was carried out by selecting the targeted m/z of 1273.5 and in a CID mode where fragmentation was induced by collision with an inert gas. Irradiation of energy required for fragmentation was performed at a standard collision energy unique to the apparatus, i.e., 35.

In order to study the fragmented pattern of the novel steviol glycoside, standard samples rebaudiosides A. D and M with known structures were subjected to MS/MS and MS³ fragmentation pattern analyses. As a result, MS/MS of the novel steviol glycoside gave data showing that the highest ion intensity appeared at the peak where the whole sugar chain attached to C-19 via an ester bond was released. This result shows the total molecular weight of the sugar chain attached to the carbon of C-19 via an ester bond.

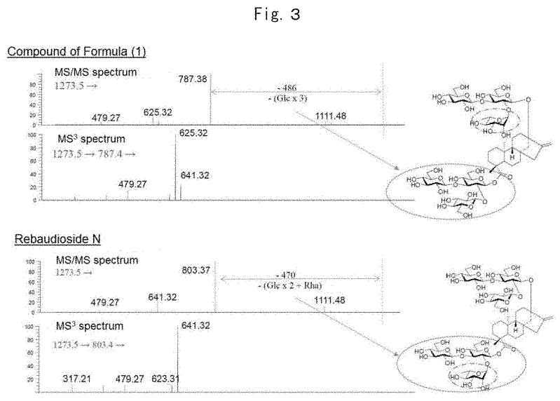

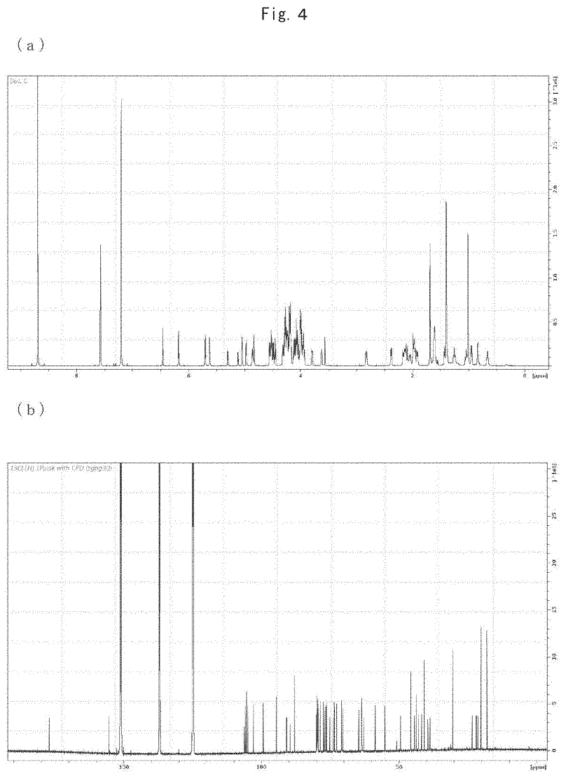

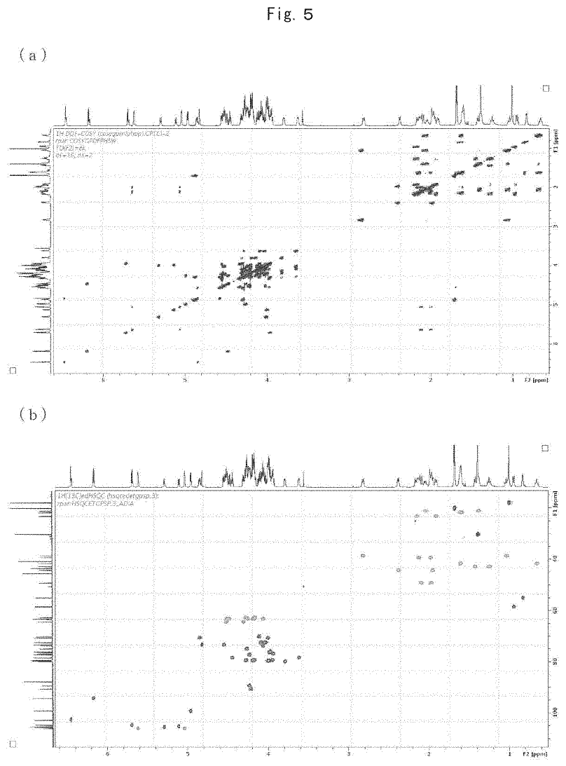

The MS/MS and MS³-fragmented mass spectra of rebaudioside N and the novel steviol glycoside are shown in FIG. 3. When the MS/MS spectra of rebaudioside N and the novel steviol glycoside having the same MS value were compared, rebaudioside N had the main peak at m/z of 803.37 corresponding to release of two glc moieties and one rha moiety. On the other hand, in the MS/MS spectrum of the novel steviol glycoside, the main peak was detected at m/z of 787.38 corresponding to release of three Glc moieties. In order to acquire further structural information, a MS' spectrum was acquired by fragmenting the main peak at m/z of 787.4 obtained by MS/MS. As a result, a spectrum having the same peak pattern as the MS³ spectrum of rebaudioside C (949.4→787.4→) was acquired. Accordingly, the sugar chain attached to C-13 was presumed to be the same as rebaudioside C. Considering that the stevia leaves of this sample also contained rebaudioside M, the sugar chain structure attached to C-19 of the novel steviol glycoside was presumed to be the same as the structure of rebaudioside M based on the potential of biosynthesis of the stevia leaves. The deduced structure is shown in FIG. 3.

(1) Outline of Synthetic Pathways

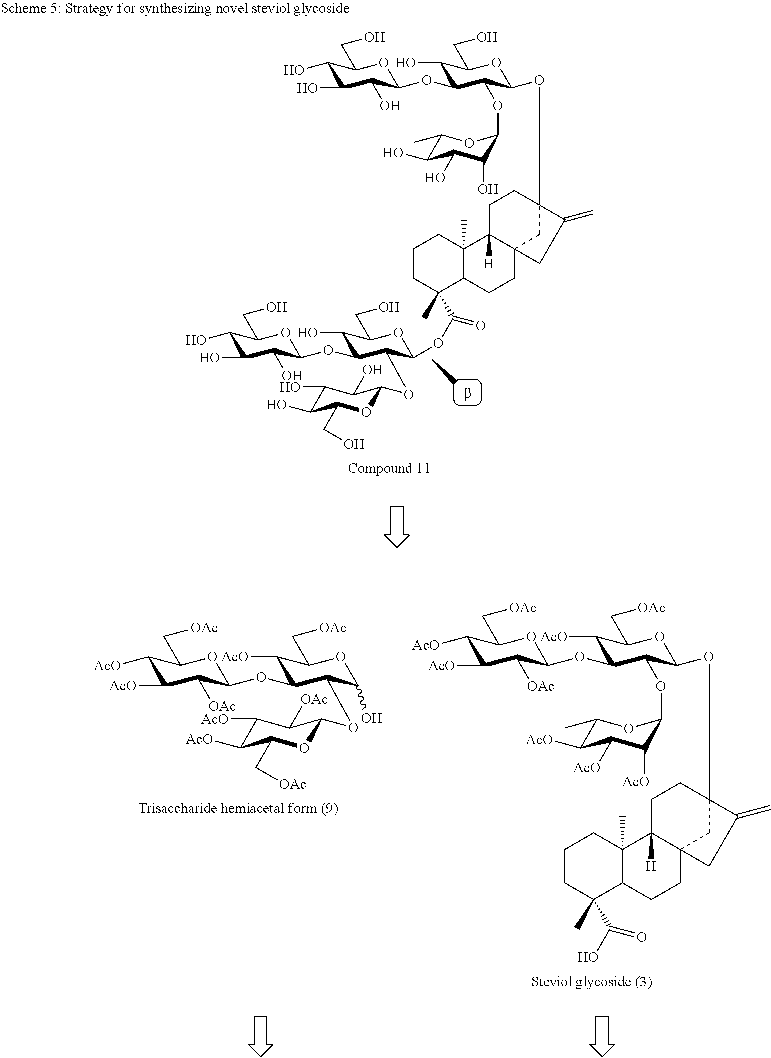

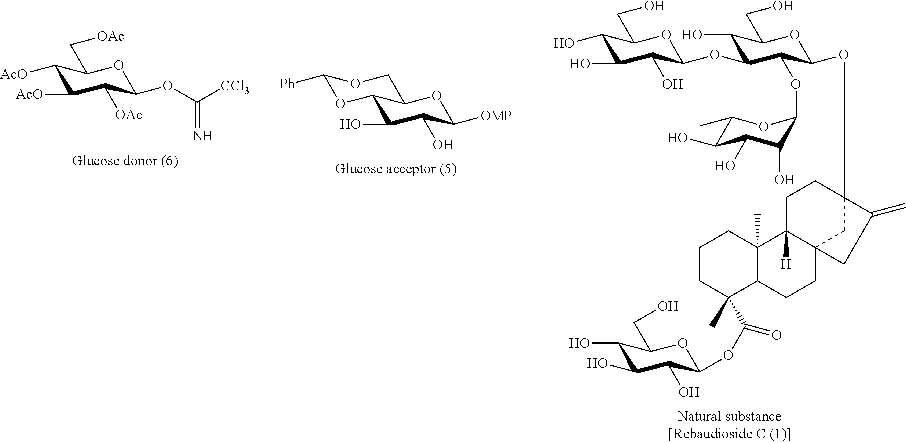

As can be appreciated from Scheme 5, for the synthesis of the novel steviol glycoside (11), the steviol glycoside (3) and the trisaccharide hemiacetal form (9) were condensed via the Mitsunobu reaction to obtain the backbone of the novel steviol glycoside (11). For synthesis of the steviol glycoside (3), a known natural substance, rebaudioside C (1), was purchased from Ark Pharm, the ester bond at C-19 of steviol was subjected to alkaline hydrolysis and then the hydroxyl groups of the sugar chain were protected with acetyl (Ac) groups to obtain the steviol glycoside (3). For synthesis of the trisaccharide hemiacetal form (9), a trisaccharide backbone was produced by condensation reaction between the appropriately protected glucose acceptor (5) and glucose donor (6), and the protecting group at the anomeric carbon of the reducing end was deprotected to give the trisaccharide hemiacetal form (9). The resulting steviol glycoside (3) and trisaccharide hemiacetal form (9) were subjected to condensation via the Mitsunobu reaction, where a reaction with good and complete β-selectivity of 47% (only the β-form) proceeded. The protecting groups of the resulting compound were deprotected, thereby obtaining the novel steviol glycoside (11).

Next, each of the synthesis steps will be described.

### (2) Synthesis of Steviol Glycoside

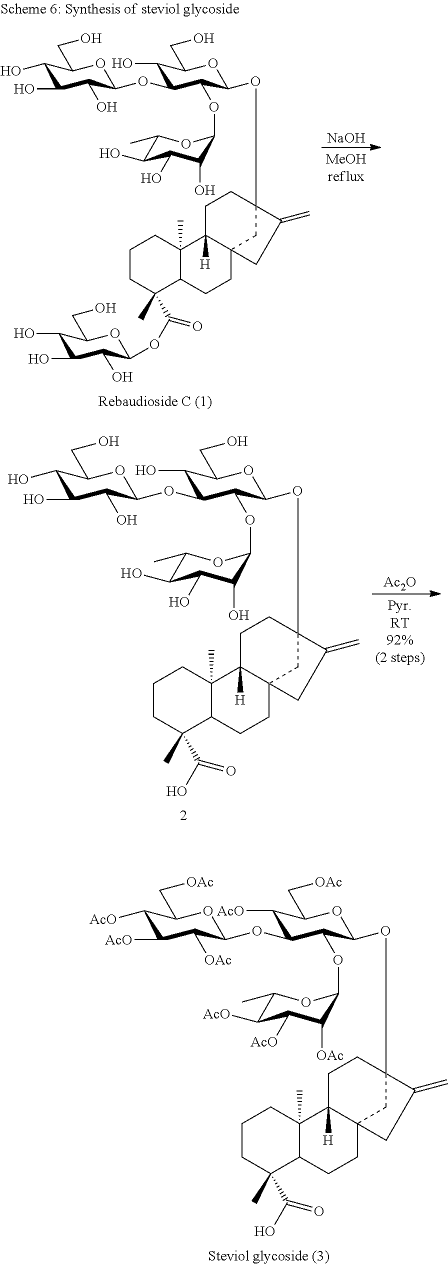

As can be appreciated from Scheme 6, for synthesis of the steviol glycoside (3), rebaudioside C (1) (1.0 g, 1.05 mmol) purchased from Ark Pharm was dissolved in methanol (10 mL) and water (10 mL), added with 4 mol/L of sodium hydroxide (2.6 mL, 10.5 mmol) at room temperature, and refluxed at 100° C. for 20 hours. The completion of the reaction was confirmed by TLC (chloroform/methanol/water=5/4/0.1, Rf value=0.9) before the reaction solution was neutralized with cation exchange resin Dowex MAC-3 hydrogen form (SIGMA-ALDRICH) (pH 7). After the resin was removed by filtration, the resultant was concentrated under a reduced pressure. The resulting syrup was dried for 18 hours by using a vacuum pump to give Compound 2 (828 mg, quant.).

Compound 2 (828 mg, 1.05 mmol) was dissolved in pyridine (20 mL), added with acetic anhydride (5 mL) at room temperature and agitated for 48 hours at room temperature. After confirming the completion of the reaction by TLC (ethyl acetate/hexane=2/1, Rf value=0.5), a saturated sodium hydrogen carbonate solution (5 mL) was added, and the reaction solution was concentrated under a reduced pressure. The resulting syrup was subjected to silica gel column chromatography and an eluate (ethyl acetate/hexane=2/1) was used to give Compound 3 (1.1 g, 92%).

[Compound 3]

¹H-NMR (CDCl₃, 400 MHz) δ 0.81 (m, 2H), 0.83-1.45 (complex, 19H), 1.39-1.91 (complex, 24H), 1.91-2.35 (s, 30H), 3.58 (m, 1H), 3.71-3.81 (complex, 4H), 3.95-4.12 (complex, 7H), 4.34-4.46 (complex, 3H), 4.56-4.66 (complex, 4H), 4.69-4.92 (complex, 7H), 5.05-5.14 (complex, 5H), 5.23-5.38 (complex, 6H), 5.45 (s, 1H); ¹³C-NMR (CDCl₁, 100 MHz) δ 15.9, 17.3, 19.1, 20.5, 20.7, 20.8, 20.9, 21.1, 21.5, 21.7, 29.1, 37.8, 38.0, 39.5, 40.7, 41.4, 42.2, 43.8, 48.4, 53.8, 56.8, 61.6, 63.0, 65.5, 66.8, 68.0, 68.6, 69.3, 69.6, 69.8, 70.5, 70.9, 71.6, 71.9, 72.4, 72.8, 73.9, 74.9, 81.3, 87.3, 96.6, 96.8, 99.2, 99.4, 125.4, 128.3, 129.1, 137.9, 151.9, 168.9, 169.2, 169.5, 169.6, 169.8, 170.1, 170.2, 170.3, 170.6, 170.9, 176.8, 183.4

### (3) Synthesis of Trisaccharide Hemiacetal Form

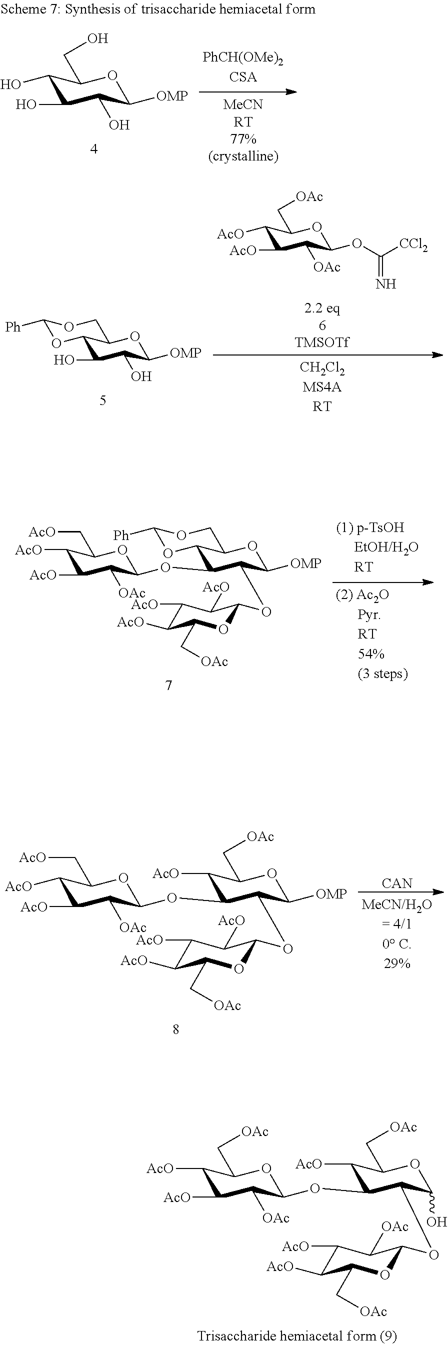

As can be appreciated from Scheme 7, for synthesis of the trisaccharide hemiacetal form (9), 4-methoxyphenyl β-D-glucopyranoside (4) (4.0 g, 13.9 mmol) purchased from Tokyo Chemical Industry was dissolved in acetonitrile (70 mL), added with benzaldehyde dimethyl acetal (3.1 mL, 20.9 mmol) and camphorsulfonic acid (323 mg, 1.39 mmol) at room temperature, and agitated at 50° C. for 18 hours. After confirming the completion of the reaction by TLC (chloroform/methanol=10/1, Rf value=0.5), the resultant was neutralized with triethylamine (I mL) (pH 8) and concentrated under a reduced pressure. The resulting residue was crystallized (ethanol) to give Compound 5 (4.0 g, 77%).

[Compound 5]

¹H-NMR (CDCl₃, 400 MHz) δ 3.58 (m, 1H, H-5), 3.65 (t, 1H, H-4), 3.78 (m, 5H, H-2, H-6, OMe), 3.92 (t, 1H, H-3), 4.38 (dd, 1H, H-6′), 4.92 (d, J=7.6 Hz, 1H, H-1), 5.57 (s, 1H CHPh), 6.84 (dd, 4H, OMePh), 7.49 (m, 5H, Ph); ¹³C-NMR (CDCl₃, 100 MHz) δ 31.1, 55.8, 66.7, 68.8, 73.4, 74.6, 80.5, 102.2, 102.5, 114.8, 118.8, 126.4, 128.5, 129.5, 136.9, 150.9, 155.9

Compound 5 (1.0 g, 2.67 mmol), 3,4,6-tetra-O-acetyl-β-D-glucosypyranosyl 2,2,2-trichloroacetimidate (6) (2.9 g, 5.88 mmol) purchased from Tokyo Chemical Industry and 4 Å molecular sieves (2.0 g) were dissolved in dichloromethane (171 mL), added with trimethylylsilyl trifluoromethanesulfonate (48 μL, 0.27 mmol) at 0° C., and agitated at room temperature for 1.5 hours. After confirming the completion of the reaction by TLC (toluene/ethyl acetate=1/1, Rf value=0.7), the resultant was neutralized with triethylamine (100 μL) (pH 8), 4 Å molecular sieves was removed by filtration, and the resultant was concentrated under a reduced pressure. The resulting syrup was subjected to silica gel column chromatography and an eluate (toluene/ethyl acetate=2/1) was used to give Compound 7.

Compound 7 (1.9 g, 1.84 mmol) was dissolved in ethanol (18 mL), added with P-toluenesulfonic acid (35 mg, 0.184 mmol) at room temperature, and agitated at 60° C. for 5.5 hours. After confirming the completion of the reaction by TLC (ethyl acetate/hexane=2/1, Rf value=0.1), the resultant was neutralized with triethylamine (5.0 mL) (pH 8) and concentrated under a reduced pressure. The resulting syrup was dissolved in pyridine (18 mL), added with acetic anhydride (347 μL, 3.68 mmol) at room temperature, and agitated at room temperature for 18 hours. After confirming the completion of the reaction by TLC (ethyl acetate/hexane=2/1, Rf value=0.7), azeotropic distillation with toluene (30 mL) was repeated for three times, and the resultant was concentrated under a reduced pressure. The resulting syrup was subjected to silica gel column chromatography and an eluate (ethyl acetatehexane=1/1→2/1) was used to give Compound 8 (1.5 g, 54%, 3 steps).

[Compound 8]

¹H-NMR (CDCl₃, 400 MHz) δ 1.94-2.17 (complex, 30H, OAc), 2.91 (m, 1H), 3.33 (m, 1H), 3.71 (m, 1H), 3.76 (m, 5H), 3.94 (t, 1H), 4.09-4.17 (complex, 5H), 4.31 (dd, 1H), 4.88 (m, 3H), 4.96-5.08 (complex, 4H), 5.18 (m, 2H), 5.26 (t, 1H), 6.84 (dd, OMePh); ¹³C-NMR (CDCl₃, 100 MHz) δ 20.6×2, 20.7×4, 20.8×2, 20.9, 21.1, 55.8, 60.3, 60.5, 61.8, 62.6, 67.6, 68.3, 71.4, 71.6, 71.9, 71.0, 72.1, 72.7, 73.1, 82.3, 98.5, 98.7×2, 114.9, 116.1, 150.1, 155.4, 168.9, 169.4×2, 169.5, 170.2, 170.3, 170.4, 170.5

Compound 8 (1.3 g, 1.26 mmol) was dissolved in acetonitrile (20 mL) and water (5.0 mL), added with cerium ammonium nitrate (1.4 g, 2.52 mmol) at 0° C. and agitated at 0° C. for 15 minutes. After confirming the completion of the reaction by TLC (ethyl acetate/hexane=2/1. Rf value=0.3), the resultant was diluted with ethyl acetate, and the organic layer was washed with water and a saturated aqueous sodium hydrogen carbonate solution and dried with magnesium sulfate. Magnesium sulfate was removed by filtration and the resultant was concentrated under a reduced pressure. The resulting syrup was subjected to silica gel column chromatography and an eluate (ethyl acetateihexane=2/1) was used to give Compound 9 (343 mg, 29%).

[Compound 9]

¹H-NMR (CDC₃, 400 MHz) δ 1.95-2.33 (complex, 55H, OAc), 3.61 (m, 6H), 3.73 (m, 1H), 3.91-4.31 (complex, 12H), 4.40 (m, 2H), 4.61 (d, J=7.6 Hz, 1H), 4.65 (d, J=7.6 Hz, 2H), 4.73 (d, J=8.0 Hz, 1H), 4.82 (d, J=8.0 Hz, 1H), 4.85-4.98 (complex, 4H), 5.01-5.21 (complex, 9H), 5.41 (d, J=3.2 Hz, 1H); ¹³C-NMR (CDCl₃, 100 MHz) δ 20.8, 20.9×3, 21.1, 21.2, 21.5, 29.4, 29.8, 61.6, 61.7×2, 61.9, 62.4, 62.5, 67.3, 67.5, 67.9, 68.1×2, 68.2, 68.3, 71.6, 71.8, 71.9×2, 71.1, 72.2, 72.3, 72.8, 73.0, 74.8, 77.4, 78.3, 81.7, 82.8, 92.2, 95.6, 98.9, 99.5, 100.1, 101.5, 125.4, 128.3, 129.1, 137.9, 168.9, 169.4, 169.5×2, 169.9, 170.0, 170.1×2, 170.3×2

### (4) Synthesis of Compound 11

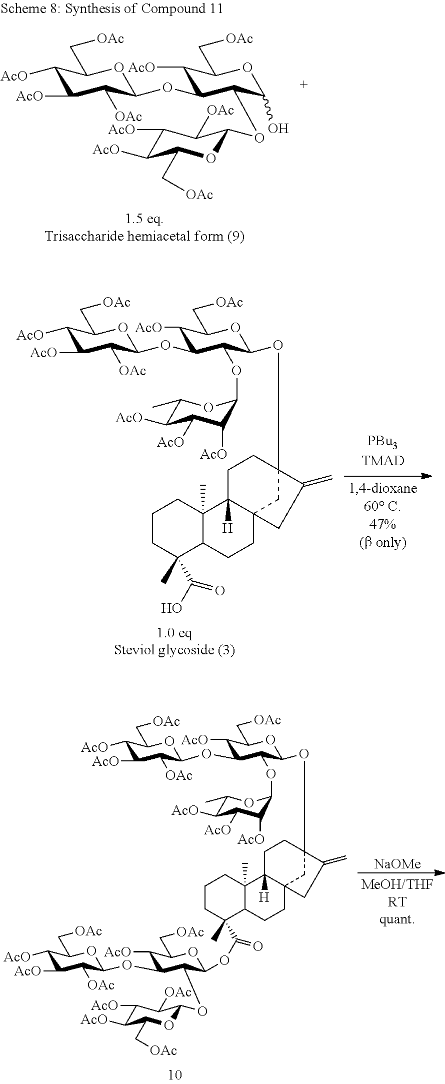

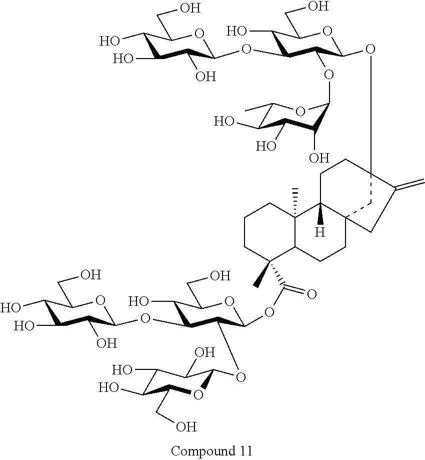

As can be appreciated from Scheme 8, for synthesis of Compound 11, Compound (9) (343 mg, 0.371 mmol) and Compound (3) (289 mg, 0.247 mmol) were dissolved in 1,4-dioxane (12 mL), added with tributyiphosphine (185 μL, 0.741 mmol) and 1,1′-azobis (N,N′-dimethylformamide) (TMAD) (128 mg, 0.741 mmol) at room temperature, and agitated at 60° C. for 18 hours. After confirming the completion of the reaction by TLC (toluene/ethyl acetate=1/2, Rf value=0.6), the resultant was diluted with ethyl acetate, and the organic layer was washed with water, a saturated aqueous sodium hydrogen carbonate solution and saturated saline, and dried with magnesium sulfate. Magnesium sulfate was removed by filtration and the resultant was concentrated under a reduced pressure. The resulting syrup was subjected to silica gel column chromatography and an eluate (toluene/ethyl acetate=1/1) was used to give Compound 10 (240 mg, 47%).

[Compound 10]

¹H-NMR (CDCl₃, 400 MHz) δ 0.50-1.05 (complex, 7H), 1.19 (d, 3H, H-6 of Rham), 1.23 (s, 3H), 1.35-2.30 (complex, 80H), 3.59 (m, 1H), 3.72 (m, 5H), 3.92-4.11 (complex, 10H), 4.21 (dd, 1H), 4.31 (dd, 1H), 4.42 (m, 3H), 4.60 (d, J=7.6 Hz, 1H), 4.71-4.95 (complex, 9H), 5.07 (m, 6H), 5.19 (t, 1H), 5.29 (m, 4H), 5.59 (d, J=7.6 Hz, 1H); ¹³C-NMR (CDCl₃, 100 MHz) δ 16.7, 17.4, 20.5, 20.7×3, 20.8, 20.9×4, 21.1×2, 21.6, 29.2, 39.5, 42.5, 44.2, 53.8, 57.4, 61.9, 66.7, 68.0, 68.3, 68.4, 68.5, 69.7, 71.1, 71.5, 71.8, 71.9, 72.0, 72.2, 72.3, 72.4, 72.9, 73.0, 75.0, 80.1, 86.8, 91.3, 96.4, 96.9, 99.2, 99.3, 99.5, 125.4, 128.3, 129.2, 152.8, 169.0, 169.1, 169.3, 169.5×2, 169.6, 170.1×2, 170.2×2, 170.5, 170.6, 170.9, 174.8

Compound (10) (220 mg, 0.106 mmol) was dissolved in methanol (2.0 mL) and THF (2.0 mL), added with sodium methoxide (0.5 M in MeOH) (0.2 mL, 0.106 mmol) at room temperature, and agitated at room temperature for 48 hours. After confirming the completion of the reaction by TLC (chloroformmethanol/water=5/4/1. Rf value=0.3), the resultant was concentrated under a reduced pressure. The resulting syrup was subjected to gel filtration column (GE Healthcare, Sephadex LH-20, ethanol) and lyophilized with water to give Compound 11 (135 mg, quant.).

[Compound 11]

¹H-NMR (pyridine-d5, 800 MHz) δ 0.68 (m, 1H), 0.86 (m, 1H), 0.97 (m, 1H), 1.04 (m, 4H), 1.28 (m, 1H), 1.43 (m, 5H), 1.63 (s, 3H), 1.70 (s, 3H), 1.93-2.20 (complex, 8H), 2.40 (d, 1H), 2.84 (d, 1H), 3.64 (m, 1H), 3.82 (m, 1H), 3.94-4.15 (complex, 13H), 4.17-4.35 (complex, 14H), 4.45-4.59 (complex, 5H), 4.88 (m, 2H), 4.99 (d, J=7.2 Hz, 1H), 5.07 (s, 1H), 5.14 (d, J=8.0 Hz, 1H), 5.32 (d, J=8.0 Hz, 1H), 5.65 (s, 1H), 5.72 (d, J=8.0 Hz, 1H), 6.20 (d, J=8.0 Hz, 1H), 6.48 (s, 1H); ¹³C-NMR (pyridine-d5, 200 MHz) 5 17.0, 19.1, 20.2, 20.9, 22.3, 29.4, 37.7, 38.5, 39.9, 40.8, 41.9, 42.7, 43.4, 44.7, 48.4, 49.8, 54.1, 57.7, 61.9, 62.4, 62.5, 62.6, 63.6, 69.4, 69.8×2, 71.6, 71.7, 72.5, 72.8, 74.1, 75.2, 75.5, 76.0, 76.4, 77.5, 77.6, 78.5×2, 78.6×2, 78.8×2, 86.9, 88.5, 89.8, 93.5, 98.4, 101.9, 103.9, 104.4, 104.8, 105.2, 154.5, 176.1

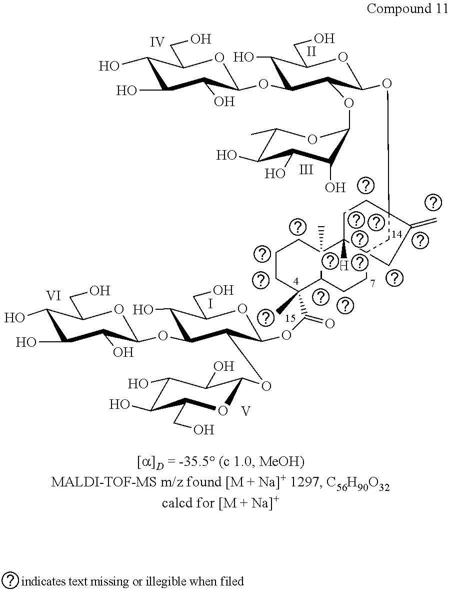

(iii) Structural determination by matching with the chemically synthesized standard product with respect to the retention time and the fragmented pattern from HPLC-high resolution MS/MS and MS³ fragmentation

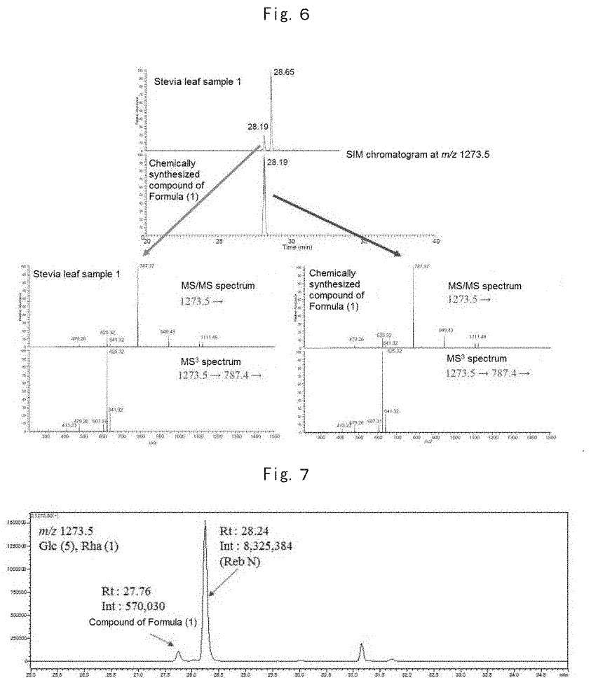

The chemically synthesized product (Compound 11) and stevia leaf liquid extracts were compared by HPLC-high resolution MS/MS and MS³-fragmentation under the same conditions as (i). As a result, the peaks of the chemically synthesized product and the stevia leaf liquid extract matched at the peak with the retention time of 28.19 minutes (FIG. 6). From this result, the novel steviol glycoside obtained from the liquid extract of the plant was confirmed to have the same structure as Compound 11.

[Biosynthesis of Novel Steviol Glycoside]

A novel steviol glycoside was generated from steviol in yeast. First, a yeast capable of coexpressing four types of stevia-derived glycosylated enzyme genes UGT85C2, UGT91D2, UGT74G1 and UGT76G1 and Arabidopsis thahana-derived UDP-rhamnose synthase gene AtRHM2 was bred.

Unless otherwise specified, the molecular biological processes employed in this example followed the methods described in Molecular Cloning (Sambrook et al., Cold Spring Harbour Laboratory Press, 2001).

In order to clone the four stevia-derived glycosylated enzyme genes, the following primer sets were synthesized to perform PCR using cDNA prepared from stevia leaves as a template.

Primer set for UGT85C2 gene amplification
CACC-NdeI-SrUGT85C2-Fw (NdeI-recognizing site
underlined):
(SEQ ID NO: 12)
5′-CACCCATATGGATGCAATGGCTACAACTGAGAA-3′
BglII-SrUGT85C2-Rv (BglII-recognizing site
underlined):
(SEQ ID NO: 13)
5′-AGATCTCTAGTTTCTTGCTAGCACGGTGATTT-3′
Primer set for UGT91D2 gene amplification
SrUGT91D2-pET15b-FW
(SEQ ID NO: 35)
5′-TGCCGCGCGGCAGCCATATGTACAACGTTACTTATCATC-3′
SrUGT91D2-pET15b-RV
(SEQ ID NO: 36)
5′-GTTAGCAGCCGGATCCTTAACTCTCATGATCGATGGCAA-3′
Primer set for UGT74G1 gene amplification
CACC-NdeI-SrUGT74G1-Fw (NdeI-recognizing site
underlined):
(SEQ ID NO: 14)
5′-CACCCATATGGCGGAACAACAAAAGATCAAGAAAT-3′
BamHI-SrUGT74G1-Rv (BamHI-recognizing site
underlined):
(SEQ ID NO: 15)
5′-GGATCCTTAAGCCTTAATTAGCTCACTTACAAATT-3′
Primer set for UGT76G1 gene amplification
CACC-NdeI-SrUGT76G1-Fw (NdeI-recognizing site
underlined):
(SEQ ID NO: 16)
5′-CACCCATATGGAAAATAAAACGGAGACCA-3′
BamHI-SrUGT76G1-Rv (BamHI-recognizing site
underlined):
(SEQ ID NO: 17)
5′-GGATCCTTACAACGATGAAATGTAAGAAACTA-3′

stevia leaf cDNA was obtained by extracting total RNA from stevia leaves using RNeasy Plant Mini kit (QIAGEN), and subjecting 0.5 μg of them to reverse transcription (RT) reaction using Random Oligo-dT primer.

The PCR reaction solution (50 μl) had the following composition: 1 μl of stevia leaf-derived cDNA, 1×KOD plus buffer (TOYOBO), 0.2 mM dNTPs, 0.4 μmol/μl of each primer, 1 mM MgSO₄ and 1 U heat resistant KOD plus polymerase. PCR reaction consisted of reaction at 95° C. for 5 minutes, followed by amplification by a total of 30 cycles of reaction at 94° C. for 0.5 minutes, 50° C. for 0.5 minutes and 68° C. for 2 minutes. Each PCR product was subjected to electrophoresis with 0.8% agarose gel and ethidium bromide staining, by which an amplification band of nearly 1.4 kb in size was obtained as presumed from each template DNA.

This PCR product was subcloned into pENTR-TOPO Directional vector (Invitrogen) according to a method recommended by the manufacturer. DNA Sequencer model 3100 (Applied Biosystems) was used for sequencing by a primer walking process with a synthesized oligonucleotide primer, thereby confirming that all of the UGT genes of interest, namely. UGT85C2, UGT91 D2, UGT74G1 and UGT76G1 were cloned.

Construction of Yeast Expression Vector

The following primer sets were designed to integrate these UGT genes and Arabidopsis thaliana-derived UDP-rhamnose synthase gene AtRHM2 (J Biol Chem 2007, Oka et. al) into a yeast expression vector.

SrUGT85C2 set
Bgl2-UGT85C2-F (BglII-recognizing site underlined):
(SEQ ID NO: 18)
5′-ACAGATCTATGGATGCAATGGCTACAACTGAGA-3′
Sal-UGT85C2-R (SalI-recognizing site underlined):
(SEQ ID NO: 19)
5′-TAGTCGACTAGTTTCTTGCTAGCACGGTGATTTC-3′
SrUGT91D2 set
NotI-UGT91DIL3-F (NotI-recognizing site
underlined):
(SEQ ID NO: 20)
5′-AAGCGGCCGCATGTACAACGTTACTTATCATCAAAATTCAAA-3′
Pac-UGT91D1L3-R (PacI-recognizing site underlined):
(SEQ ID NO: 21)
5′-CGTTAATTAACTCTCATGATCGATGGCAACC-3′
SrUGT74G1 set
Not-UGT74G1-F (NotI-recognizing site underlined):
(SEQ ID NO: 22)
5′-AAGCGGCCGCATGGCGGAACAACAAAAGATCAAG-3′
Pac-UGT74G1-R (PacI-recognizing site underlined):
(SEQ ID NO: 23)
5′-CGTTAATTAAGCCTTAATTAGCTCACTTACAAATTCG-3′
SrUGT76G1 set
Bam-UGT76G1-F (BamHI-recognizing site underlined):
(SEQ ID NO: 24)
5′-AAGGATCCATGGAAAATAAAACGGAGACCACCG-3′
Sal-UGT76G1-R (SalI-recognizing site underlined):
(SEQ ID NO: 25)
5′-GCGTCGACTTACAACGATGAAATGTAAGAAACTAGAGACTCTAA-3′
AtRHM2 set
Bam-AtRHM2-F (BamHI-recognizing site underlined):
(SEQ ID NO: 26)
5′-GGATCCATGGATGATACTACGTATAAGCCAAAG-3′
Xho-AtRHM2-R (XhoI-recognizing site underlined):
(SEQ ID NO: 27)
5′-CTCGAGTTAGGTTCTCTTGTTTGGTTCAAAGA-3′

The combinations of templates and primers, namely, template UGT85C2 and SrUGT85C2 set, template UGT91D2 and SrUGT91D2 set, template UGT74G1 and SrUGT74G1 set, template UGT76G1 and SrUGT76G1 set, and template AtAHM2 and AtAHM2 set, were used for PCR amplification using heat resistant KOD DNA polymerase (TOYOBO) and introduction of the restriction enzyme sites at both ends of each ORE The resulting DNA fragment was subcloned using Zero Blunt-TOPO PCR cloning kit (Invitrogen), and sequenced with DNA Sequencer model 3100 (Applied Biosystems) by a primer walking process with a synthesized oligonucleotide primer to confirm that each of the UGT genes of interest was cloned.

In order to express the above-described genes in yeasts by using pESC yeast expression system (Stratagene), the following expression vectors were constructed.

(1) Construction of Plasmid pESC-URA-UGT56

UGT85C2 was cleaved with restriction enzymes BglII and SalI, and linked to vector pESC-URA (Stratagene) that had been cleaved with restriction enzymes BamHI and Sail to give plasmid pESC-URA-UGT-5. This plasmid pESC-URA-UGT-5 was cleaved with restriction enzymes NotI and PacI while UGT91 D2 was also cleaved with restriction enzymes NotI and PacI. The resultants were linked to give pESC-URA-UGT56.

(2) Construction of Plasmid pESC—HIS-UGT78

UGT76G1 was cleaved with restriction enzymes BamHI and SalI, and linked to vector pESC-HIS (Stratagene) that had been cleaved with the same restriction enzymes to give plasmid pESC—HIS-UGT-8. This plasmid pESC—HIS-UGT-8 was cleaved with restriction enzymes NotI and PacI while UGT74G1 was also cleaved with NotI and PacI. The resultants were linked to give pESC—HIS-UGT78.

(3) Construction of Plasmid pESC-TRP-AtRHM2

AtAHM2 was cleaved with restriction enzymes BamHI and XhoI while vector pESC-TRP (Stratagene) was cleaved with the same restriction enzymes. The resultants were linked to give plasmid pESC-TRP-AtAHM2.

Transformation of Yeast

Plasmids shown in Table 2 were introduced into Saccharomyces cerevisiae YPH499 strain (ura3-52 lys2-801^{amber}ade2-101^{ochre}trp1-Δ63 his3-Δ200 leu2-Δ1a) as a host by lithium acetate technique. As transformed strains, those that survived in a SC-Trp-Ura-His agar medium (6.7 g of yeast nitrogen base without amino acids, 20 g of glucose, 1.3 g of amino acid powder mix-Trp-Ura-His and 20 g of Bacto agar per 1 L) were selected.

TABLE 2

| Transformed strain | Plasmids introduced | Genes introduced |
|---|---|---|
| A-5678 | PESC-URA-UGT-56 | SrUGT85C2, SrUGT91D2 |
| | pESC-HIS-UGT-78 | SrUGT74G1, SrUGT76G1 |
| | pESC-TRP-AtAHM2 | AtAHM2 |

Here, the amino acid powder mix-Trp-Ura-His was prepared by mixing adenine sulfate (2.5 g), L-arginine hydrochloride (1.2 g), L-aspartic acid (6.0 g), L-glutamic acid (6.0 g), L-leucine (3.6 g), L-lysine (1.8 g), L-methionine (1.2 g), L-phenylalanine (3.0 g), L-serine (22.5 g), L-threonine (12 g), L-tyrosine (1.8 g) and L-valine (9.0 g).

Induction and Analysis of Expression of Transgene

The resulting transformed strain was cultured as follows.

First, for preliminary culture, each transformed strain was seeded in 10 ml of a SC-Trp-Ura-His liquid medium (SC-Trp-Ura-His agar medium without Bacto agar) and shake cultured at 3(0C for a day. Subsequently, for main culture, 1 ml of the preliminary culture solution was seeded into 10 ml of SG-Trp-Ura-His liquid medium (6.7 g of yeast nitrogen base without amino acids, 20 g of galactose, and 1.3 g of amino acid powder mix-Trp-Ura-His per 1 L) and shake cultured at 30° C. for two days.

In order to confirm whether or not the introduced gene was expressed in the transformed strain, cells were harvested from the culture solution to purify total RNA with RNeasy Mini Kit.

With 1 g±g of total RNA, cDNA was synthesized using SuperScript II reverse transcriptase (Thermo Fischer Scientific) and random hexamer as a primer.

In order to confirm expression of the transgene, the following primers were prepared.

For confirming expression of UGT85C2
UGT85C2-r1:
(SEQ ID NO: 28)
5′-CAAGTCCCCAACCAAATTCCGT-3′
For confirming expression of UGT91D2
UGT91D1L3-r1:
(SEQ ID NO: 29)
5′-CACGAACCCGTCTGGCAACTC-3′
For confirming expression of UGT74G1
UGT74G1-r1:
(SEQ ID NO: 30)
5′-CCCGTGTGATTTCTTCCACTTGTTC-3′
For confirming expression of UGT76G1
UGT76G1-r1:
(SEQ ID NO: 31)
5′-CAAGAACCCATCTGGCAACGG-3′
For confirming expression of AtAHM2
AtAHM2-r1
(SEQ ID NO: 32)
5′-GCTTTGTCACCAGAATCACCATT-3′
GAL10p region (promoter region)
PGAL10-f3:
(SEQ ID NO: 33)
5′-GATTATTAAACTTCTTTGCGTCCATCCA-3′
GAL1p region (promoter region)
PGAL1-f3:
(SEQ ID NO: 34)
5′-CCTCTATACTTTAACGTCAAGGAGAAAAAACC-3′

Expression of each transgene was confirmed by performing PCR by using ExTaq (Taraka Bio) with the following combination of primers and the previously synthesized cDNA as a template and subjecting the resulting product to agarose gel electrophoresis.

UGT85C2: UGT85C2-r1 (SEQ ID NO:28) and PGAL1-f3 (SEQ ID NO:34)

UGT91D2 or UGT91D2L3: UGT91DIL3-r1 (SEQ ID NO:29) and PGAL10-f3 (SEQ ID NO:33)

UGT74G1: UGT74G1-r1 (SEQ ID NO:30) and PGAL1-f3 (SEQ ID NO:34)

UGT76G1: UGT76G1-r1 (SEQ ID NO:31) and PGAL10-f3 (SEQ ID NO:33)

AtAHM2: AtAHM2-r1 (SEQ ID NO:32) and PGAL10-f3 (SEQ ID NO:33) Accordingly, expression of the transgene in the transformed strain was confirmed.

Production of novel steviol glycoside Culturing was performed under the same conditions as described above except

that 0.5 μg or 2 μg of steviol (ChromaDex Inc.) was added to the liquid medium for the main culture per 1 ml of the medium. After culturing, the culture solution was separated into supernatant and cells by centrifugation. The culture supernatant was washed with acetonitrile, then subjected to a water-equilibrated Sep-Pak C18 column, washed with 20% acetonitrile, eluted with 80% acetonitrile, dried to solidify, and then dissolved in a small amount of 80% acetonitrile to prepare a glycoside sample. This glycoside sample was subjected to the following analysis.

Analysis by LC-MS

An analysis by LC-MS was carried out as described in the example under “Isolation of novel steviol glycoside”.

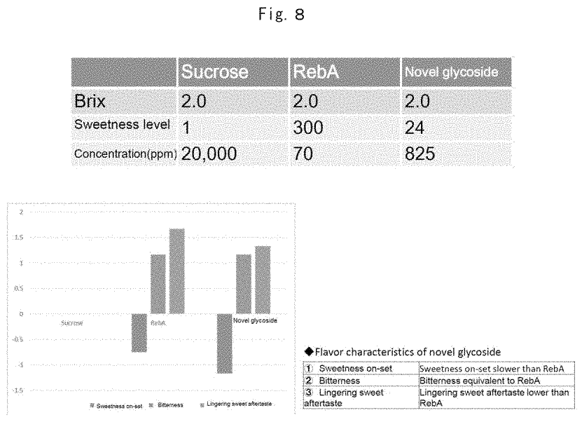

The result is shown in FIG. 7. Generation of the novel steviol glycoside in A-5678 strain was confirmed. This result corresponds to that for the steviol glycoside resulting from the above-described chemical synthesis.

Evaluation of Sweetness Level of Novel Steviol Glycoside

In order to evaluate the sweetness level of the novel steviol glycoside, samples were prepared by adding sucrose to pure water to give Brix of 0.5 to 3 in 0.5 increments. A sample was prepared by adding the novel steviol glycoside to pure water to 415 ppm.

Evaluation was conducted by selecting the sucrose-added sample having sweetness intensity equivalent to that of the sample added with the novel steviol glycoside, where sensory evaluation was conducted by panelists trained about sensory attributes of sweeteners (5 members). As a result, the sample prepared by adding the novel glycoside was found to have sweetness equivalent to that of the sucrose-added sample with Brix of 1. Therefore, the novel steviol glycoside of the invention was found to have a sweetness level of 24 with respect to sucrose.

Sensory Evaluation of Novel Steviol Glycoside

In order to evaluate the taste quality of various steviol glycosides, Reb. A and the novel steviol glycoside were added to pure water at amounts indicated in FIG. 8 to prepare beverage samples. All of the beverage samples were adjusted to have final Brix of 2 in terms of sucrose, provided that the sweetness levels were RebA: 300 and novel glycoside: 24.

The resulting beverage samples were subjected to sensory evaluation for rating attributes, namely, sweetness on-set, bitterness and lingering sweet aftertaste. Panelists trained about sensory attributes of sweeteners (5 members) evaluated based on the following evaluation criteria. Very weak (−3), weak (−2), slightly weak (−1), normal (0), slightly strong (+1), strong (+2) and very strong (+3).

As a result of the sensory evaluation, the novel steviol glycoside was found to have equal bitterness and shorter lingering sweet aftertaste as compared to the conventional sweetener Reb.A.

Evaluation of Flavor Controlling Agent Containing Novel Steviol Glycoside for Improving Lingering Aftertaste

(1) Measurement of Sweetness Level of Sweetener Targeted for Improvement of Lingering Aftertaste

Prior to evaluation of the flavor controlling agent, the sweetness level of the sweetener targeted for improvement of lingering aftertaste was measured. Reb.A (purity 100%) and Reb.D (purity 97%) were used as the sweeteners. Reb.A and Reb.D were dissolved in water in amounts indicated in the table below to prepare aqueous solutions. In the meantime, standard aqueous solutions having Brix of 5-7 were prepared using sucrose (sugar), for which panelists trained about sensory attributes of sweeteners (6 members) evaluated as to which standard aqueous solution had the corresponding sweetness to that of the aqueous Reb.A solution and the aqueous Reb.D solution. The results are shown in the table below.

TABLE 3

| | Aqueous Reb. | Aqueous Reb. |
|---|---|---|
| | A solution | D solution |
| Concentration of sweetener in | 23.3 mg/100 ml | 28 mg/100 ml |
| aqueous solution | | |
| Brix of corresponding standard | 5.53 | 5.96 |
| aqueous solution (average | | |
| evaluation value by 5 panelists) | | |
| Sweetness fold | 237 | 213 |

From the above results, evaluations in the following test were conducted provided that the sweetness fold of Reb. A was 237-fold and the sweetness fold of Reb.D was 213-fold. Here, the sweetness fold of the novel steviol glycoside used for evaluation was 24-fold as described above.

(2) Evaluation of Effect of Improving Lingering Aftertaste of Reb.A

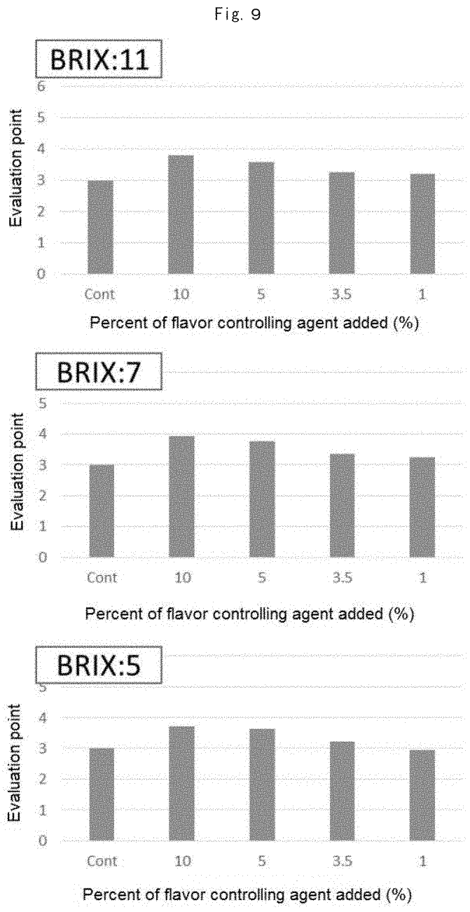

Three-level aqueous solutions with Brix of 5, 7 and 11 were used to evaluate the effect of the flavor controlling agent of the present invention to improve lingering aftertaste of Reb.A. First, three-level aqueous solutions with Brix of 5, 7 and 11 were generated provided that the sweetness level of Reb.A was 237-fold. The added amounts of the flavor controlling agent made of the novel steviol glycoside of the present invention were 1, 3.5, 5 and 10 mass % in proportion based on the mass of added Reb.A. A control sample (Cont) was added with Reb.A only and was not added with the flavor controlling agent of the present invention. Panelists trained about sensory attributes of sweeteners (7 members) conducted a numerical evaluation in which the maximum improvement of lingering aftertaste was set to 6 points while Cont was set to 3 points. The more the lingering aftertaste was improved, the higher the point was. The average evaluation points are shown in the graphs in FIG. 9.

(3) Evaluation of Effect of Improving Lingering Aftertaste of Reb.D

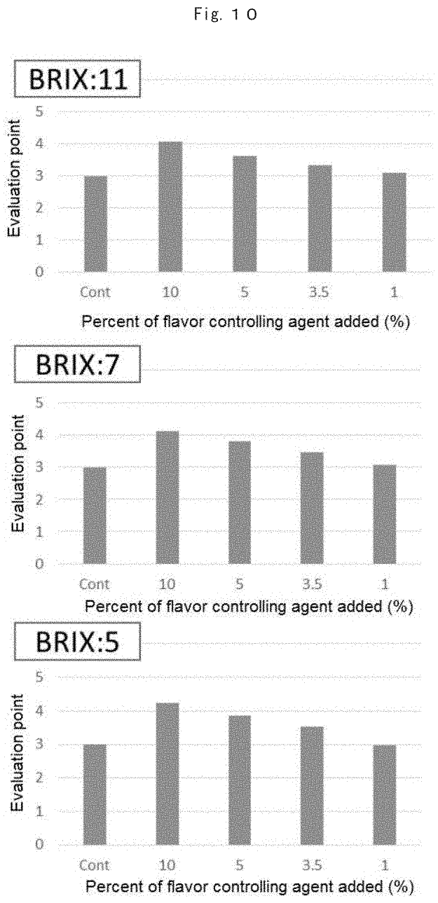

Three-level aqueous solutions with Brix of 5, 7 and 11 were used to evaluate the effect of the flavor controlling agent of the present invention to improve lingering aftertaste of Reb.D. First, three-level aqueous solutions with Brix of 5, 7 and 11 were generated provided that the sweetness level of Reb.D was 213-fold. The added amounts of the flavor controlling agent made of the novel steviol glycoside of the present invention were 1, 3.5, 5 and 10 mass % in proportion based on the mass of added Reb.D. A control sample (Cont) was added with Reb.D only and was not added with the flavor controlling agent of the present invention. Panelists trained about sensory attributes of sweeteners (7 members) conducted a numerical evaluation in which the maximum improvement of lingering aftertaste was set to 6 points while Cont was set to 3 points. The more the lingering aftertaste was improved, the higher the point was. The average evaluation points are shown in the graphs in FIG. 10.

Evaluation of Flavor Controlling Agent Containing Novel Steviol Glycoside for Enhancing Sweetness

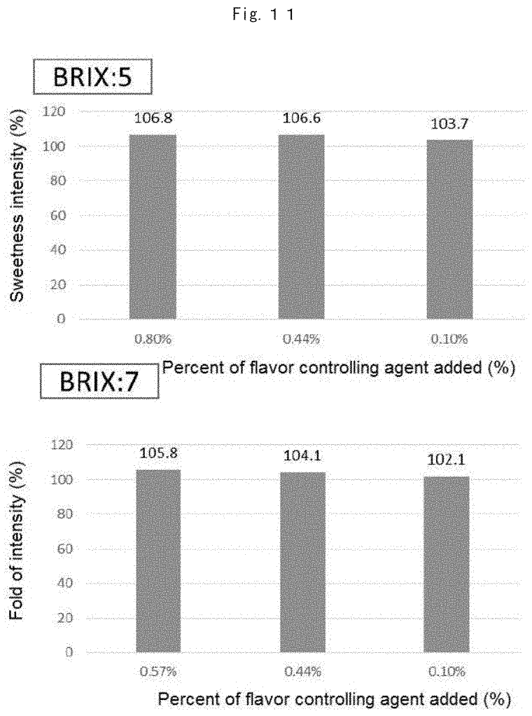

Two-level aqueous solutions with Brix of 5 and 7 were used to evaluate the effect of the flavor controlling agent of the present invention to enhance sweetness with respect to sugar (sucrose). First, two-level aqueous solutions with Brix of 5 and 7 were generated using sugar. The added amounts of the flavor controlling agent made of the novel steviol glycoside of the present invention were 0.10, 0.44 and 0.80 mass % in proportion in terms of the amount of the sugar added for Brix of 5, and 0.10, 0.44 and 0.57 mass % in proportion for Brix of 7. Panelists trained about sensory attributes of sweeteners (7 members) evaluated based on the rate of the sweetness intensity. The results are shown in graphs in FIG. 11. The vertical axes (sweetness intensity) of the graphs represent the rates of sweetness intensity actually sensed by the panelists with respect to the total sweetness level of the sugar and the flavor controlling agent (calculated provided that the sweetness level of the flavor controlling agent had a sweetness fold of 24). For all samples, a sweetness enhancement effect of about 2-7% was observed.

1. A compound represented by Formula (1): 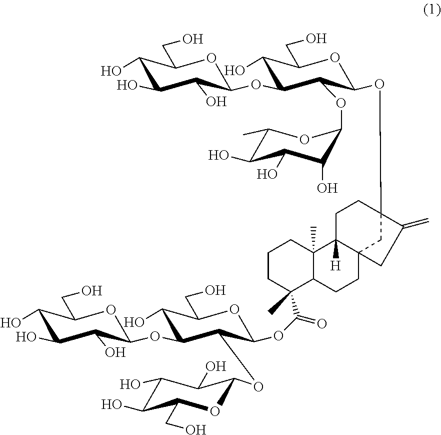
or a derivative, a salt or a hydrate thereof.

2. The compound according to claim 1, or a derivative, a salt or a hydrate thereof, wherein the compound or a derivative, a salt or a hydrate thereof is a plant-derived product, a chemically synthesized product or a biosynthetic product.

3. A sweetener composition comprising the compound according to claim 1, or a derivative, a salt or a hydrate thereof.

4. The sweetener composition according to claim 3, further comprising one or more types of steviol glycoside selected from the group consisting of rebaudioside A, rebaudioside B, rebaudioside C, rebaudioside D, rebaudioside E, rebaudioside F, rebaudioside I, rebaudioside J, rebaudioside K, rebaudioside N, rebaudioside M, rebaudioside O, rebaudioside Q, rebaudioside R, dulcoside A, rubusoside, steviol, steviol monoside, steviol bioside and stevioside.

5. A food or beverage comprising the compound according to claim 1, or a derivative, a salt or a hydrate thereof.

6. The food or beverage according to claim 5, which is a beverage.

7. A plant comprising the compound according to claim 1, or a derivative, a salt or a hydrate thereof.

8. An extract of the plant according to claim 7.

9. A food or beverage comprising the plant according to claim 7.

10. The food or beverage according to claim 9, which is a beverage.

11. A method for producing the compound of claim 1, comprising the steps of:
(A) synthesizing a compound represented by Formula (3) below: 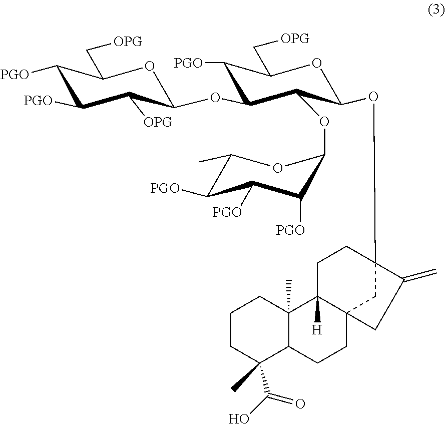
(wherein, PGs each independently represents a protecting group)
from rebaudioside C represented by Formula (2) below: 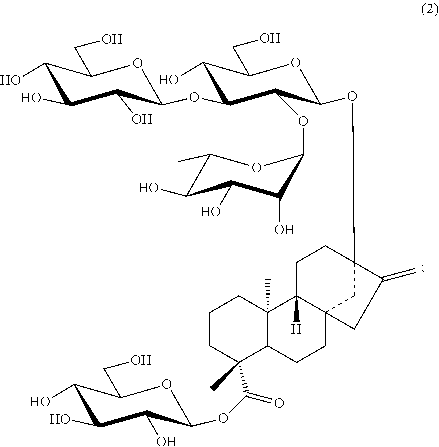
and
(B) synthesizing a compound represented by Formula (4) below: 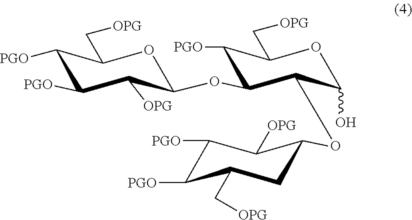
(wherein, PGs each independently represents a protecting group) from a glucopyranoside derivative.

12. The method according to claim 1, comprising a step of allowing reaction between the compound represented by Formula (3) above and the compound represented by Formula (4) above in the presence of a phosphine reagent and an azo compound to obtain a compound represented by Formula (5) below: 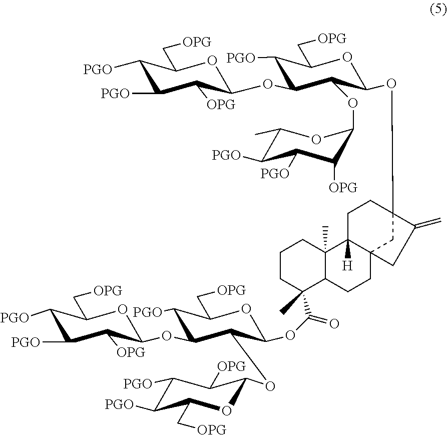
(wherein, PGs each independently represents a protecting group).

13. The method according to claim 12, wherein the yield of the step of obtaining the compound represented by Formula (5) above is 40% or more.

14. Use of the compound according to claim 1, or a derivative,
a salt or a hydrate thereof as a sweetener.

15. A method for producing the compound according to claim 1, the method characterized by use of a non-human transformant that has been introduced with at least one of polynucleotides (a) to (g) below:
(a) a polynucleotide containing the nucleotide sequence of SEQ ID NO:1, a polynucleotide containing a nucleotide sequence having 90% or higher identity with the nucleotide sequence of SEQ ID NO: 1, or a polynucleotide coding for a protein that has 90% or higher identity with the amino acid sequence of SEQ ID NO:2 and that has an activity of adding glucose to the hydroxyl group at C-13 of the steviol glycoside;
(b) a polynucleotide containing the nucleotide sequence of SEQ ID NO:3, a polynucleotide containing a nucleotide sequence having 90% or higher identity with the nucleotide sequence of SEQ ID NO:3, or a polynucleotide coding for a protein that has 90% or higher identity with the amino acid sequence of SEQ ID NO:4 and that has an activity of adding glucose to the carboxylic acid at C-19 of the steviol glycoside;
(c) a polynucleotide containing the nucleotide sequence of SEQ ID NO:5, a polynucleotide containing a nucleotide sequence having 90% or higher identity with the nucleotide sequence of SEQ ID NO:5, or a polynucleotide coding for a protein that has 90% or higher identity with the amino acid sequence of SEQ ID NO:6 and that has an activity of adding rhamnose to glucose attached to C-13 of the steviol glycoside via a 1→2 linkage;
(d) a polynucleotide containing the nucleotide sequence of SEQ ID NO:7, a polynucleotide containing a nucleotide sequence having 90% or higher identity with the nucleotide sequence of SEQ ID NO:7, or a polynucleotide coding for a protein that has 90% or higher identity with the amino acid sequence of SEQ ID NO:8 and that has an activity of adding glucose to C-3 of glucose at C-13 of the steviol glycoside via a 1→3 linkage;
(e) a polynucleotide containing the nucleotide sequence of SEQ ID NO:5, a polynucleotide containing a nucleotide sequence having 90% or higher identity with the nucleotide sequence of SEQ ID NO:5, or a polynucleotide coding for a protein that has 90% or higher identity with the amino acid sequence of SEQ ID NO:6 and that has an activity of adding glucose to glucose at C-19 of the steviol glycoside via a 1→2 linkage;
(f) a polynucleotide containing the nucleotide sequence of SEQ ID NO:7, a polynucleotide containing a nucleotide sequence having 90% or higher identity with the nucleotide sequence of SEQ ID NO:7, or a polynucleotide coding for a protein that has 90% or higher identity with the amino acid sequence of SEQ ID NO:8 and that has an activity of adding glucose to glucose at C-19 of the steviol glycoside via a 1→3 linkage; and
(g) a polynucleotide containing the nucleotide sequence of SEQ ID NO:9, a polynucleotide containing a nucleotide sequence having 90% or higher identity with the nucleotide sequence of SEQ ID NO:9, or a polynucleotide coding for a protein that has 90% or higher identity with the amino acid sequence of SEQ ID NO: 10 and that has an activity of generating UDP-rhamnose from UDP-glucose.

16. The method according to claim 15, wherein the non-human transformant is a yeast.

17. The method according to claim 15, wherein the non-human transformant is cultured in a medium containing steviol.

18. A flavor controlling agent comprising the compound, or a derivative, a salt or a hydrate thereof according to claim 1.


SEQUENCE LISTING (36 sequences)

SEQ ID NO: 1 - DNA, 1446 bp, Stevia rebaudiana
  CDS (1)..(1446)
atggatgcaatggctacaactgagaagaaaccacacgtcatcttcataccatttccagcacaaagccacattaaagccatgctcaaactagcacaacttctccaccacaaaggactccagataaccttcgtcaacaccgacttcatccacaaccagtttcttgaatcatcgggcccacattgtttggacggttcaccgggtttccggttcgaaaccatcccggatggtgtttctcacagtccggaagcgagcatcccaatcagagaatcactcttgagatccattgaaaccaacttcttggatcgtttcattgatcttgtaaccaaacttccggatcctccgacttgtattatctcagatgggttcttgtcggttttcacaattgacgctgcaaaaaagcttggaattccggtcatgatgtattggacacttgctgcctgtgggttcatgggtttttaccatattcattctctcattgagaaaggatttgcaccacttaaagatgcaagttacttgacaaatgggtatttggacaccgtcattgattgggttccgggaatggaaggcatccgtctcaaggatttcccgctggactggagcactgacctcaatgacaaagttttgatgttcactacagaagctcctcaaaggtcacacaaggtttcacatcatattttccacacgttcgatgagttggagcctagtattataaaaactttgtcattgaggtataatcacatttacaccatcggcccactgcaattacttcttgatcaaatacccgaagagaaaaagcaaactggaattacgagtctccatggatacagtttagtaaaagaagaaccagagtgtttccagtggcttcagtctaaagaaccaaattccgtcgtttatgtaaattttggaagtactacagtaatgtctttagaagacatgacggaatttggttggggacttgctaatagcaaccattatttcctttggatcatccgatcaaacttggtgataggggaaaatgcagttttgccccctgaacttgaggaacatataaagaaaagaggctttattgctagctggtgttcacaagaaaaggtcttgaagcacccttcggttggagggttcttgactcattgtgggtggggatcgaccatcgagagcttgtctgctggggtgccaatgatatgctggccttattcgtgggaccagctgaccaactgtaggtatatatgcaaagaatgggaggttgggctcgagatgggaaccaaagtgaaacgagatgaagtcaagaggcttgtacaagagttgatgggagaaggaggtcacaaaatgaggaacaaggctaaagattggaaagaaaaggctcgcattgcaatagctcctaacggttcatcttctttgaacatagacaaaatggtcaaggaaatcaccgtgctagcaagaaactag

SEQ ID NO: 2 - protein, 481 aa, Stevia rebaudiana
MDAMATTEKKPHVIFIPFPAQSHIKAMLKLAQLLHHKGLQITFVNTDFIHNQFLESSGPHCLDGSPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 3 - DNA, 1383 bp, Stevia rebaudiana
  CDS (1)..(1383)
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagagcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaa

SEQ ID NO: 4 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPELQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 5 - DNA, 1458 bp, Stevia rebaudiana
  CDS (1)..(1458)
atgtacaacgttacttatcatcaaaattcaaaagcaatggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggctggttcttaagggatctgattgtttgctttccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggttttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 6 - protein, 485 aa, Stevia rebaudiana
MYNVTYHQNSKAMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 7 - DNA, 1377 bp, Stevia rebaudiana
  CDS (1)..(1377)
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgaccgcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 8 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRPLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 9 - DNA, 2004 bp, Arabidopsis thaliana
  CDS (1)..(2004)
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 10 - protein, 667 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 11 - DNA, 60 bp, Stevia rebaudiana
gagtaaaatctataacgacactaaggtggaaaaagaatatgtaagccaattcgtagactt

SEQ ID NO: 12 - DNA, 33 bp, Artificial
  Artificial nucleic acid
cacccatatggatgcaatggctacaactgagaa

SEQ ID NO: 13 - DNA, 32 bp, Artificial
  Artificial nucleic acid
agatctctagtttcttgctagcacggtgattt

SEQ ID NO: 14 - DNA, 35 bp, Artificial
  Artificial nucleic acid
cacccatatggcggaacaacaaaagatcaagaaat

SEQ ID NO: 15 - DNA, 35 bp, Artificial
  Artificial nucleic acid
ggatccttaagccttaattagctcacttacaaatt

SEQ ID NO: 16 - DNA, 29 bp, Artificial
  Artificial nucleic acid
cacccatatggaaaataaaacggagacca

SEQ ID NO: 17 - DNA, 32 bp, Artificial
  Artificial nucleic acid
ggatccttacaacgatgaaatgtaagaaacta

SEQ ID NO: 18 - DNA, 33 bp, Artificial
  Artificial nucleic acid
acagatctatggatgcaatggctacaactgaga

SEQ ID NO: 19 - DNA, 34 bp, Artificial
  Artificial nucleic acid
tagtcgactagtttcttgctagcacggtgatttc

SEQ ID NO: 20 - DNA, 42 bp, Artificial
  Artificial nucleic acid
aagcggccgcatgtacaacgttacttatcatcaaaattcaaa

SEQ ID NO: 21 - DNA, 31 bp, Artificial
  Artificial nucleic acid
cgttaattaactctcatgatcgatggcaacc

SEQ ID NO: 22 - DNA, 34 bp, Artificial
  Artificial nucleic acid
aagcggccgcatggcggaacaacaaaagatcaag

SEQ ID NO: 23 - DNA, 37 bp, Artificial
  Artificial nucleic acid
cgttaattaagccttaattagctcacttacaaattcg

SEQ ID NO: 24 - DNA, 33 bp, Artificial
  Artificial nucleic acid
aaggatccatggaaaataaaacggagaccaccg

SEQ ID NO: 25 - DNA, 44 bp, Artificial
  Artificial nucleic acid
gcgtcgacttacaacgatgaaatgtaagaaactagagactctaa

SEQ ID NO: 26 - DNA, 33 bp, Artificial
  Artificial nucleic acid
ggatccatggatgatactacgtataagccaaag

SEQ ID NO: 27 - DNA, 32 bp, Artificial
  Artificial nucleic acid
ctcgagttaggttctcttgtttggttcaaaga

SEQ ID NO: 28 - DNA, 22 bp, Artificial
  Artificial nucleic acid
caagtccccaaccaaattccgt

SEQ ID NO: 29 - DNA, 21 bp, Artificial
  Artificial nucleic acid
cacgaacccgtctggcaactc

SEQ ID NO: 30 - DNA, 25 bp, Artificial
  Artificial nucleic acid
cccgtgtgatttcttccacttgttc

SEQ ID NO: 31 - DNA, 21 bp, Artificial
  Artificial nucleic acid
caagaacccatctggcaacgg

SEQ ID NO: 32 - DNA, 23 bp, Artificial
  Artificial nucleic acid
gctttgtcaccagaatcaccatt

SEQ ID NO: 33 - DNA, 28 bp, Artificial
  Artificial nucleic acid
gattattaaacttctttgcgtccatcca

SEQ ID NO: 34 - DNA, 32 bp, Artificial
  Artificial nucleic acid
cctctatactttaacgtcaaggagaaaaaacc

SEQ ID NO: 35 - DNA, 39 bp, Artificial
  Artificial nucleic acid
tgccgcgcggcagccatatgtacaacgttacttatcatc

SEQ ID NO: 36 - DNA, 39 bp, Artificial
  Artificial nucleic acid
gttagcagccggatccttaactctcatgatcgatggcaa